(19) United States
(12) Patent Application Publication
(10) Pub. No.: US 2022/0096664 A1
(43) Pub. Date: Mar. 31, 2022

(54) STABILIZED FORMULATIONS CONTAINING IODINATED CONTRAST AGENTS AND CYCLODEXTRINS

(71) Applicant: Verrow Pharmaceuticals, Inc., San Diego, CA (US)

(72) Inventor: Gerold L. Mosher, Kansas City, MO (US)

(21) Appl. No.: 17/369,727

(22) Filed: Jul. 7, 2021

Related U.S. Application Data
(63) Continuation of application No. 13/787,495, filed on Mar. 6, 2013, now abandoned.
(60) Provisional application No. 61/652,993, filed on May 30, 2012.

(51) Int. Cl. A61K 49/04; A61K 47/69

(52) U.S. Cl. CPC A61K 49/0438; A61K 47/6951; A61K 49/0447

(57) ABSTRACT

The invention encompasses compositions containing an iodinated contrast agent and a substituted cyclodextrin wherein the cyclodextrin stabilizes the contrast agent against degradation by ultraviolet or visible light exposure.

### CROSS-REFERENCE TO RELATED APPLICATIONS

This application is a continuation of U.S. patent application Ser. No. 13/787,495 filed Mar. 6, 2013, which claims the benefit of U.S. Provisional Application Ser. No. 61/652,993 filed May 30, 2012, the entire contents of each of which are incorporated herein by reference.

### FIELD OF THE INVENTION

The invention encompasses liquid formulations comprising an iodinated contrast agent or a salt thereof and a substituted cyclodextrin, wherein the cyclodextrin provides improved chemical stability of the contrast agent when exposed to ultraviolet or visible light irradiation.

### BACKGROUND OF THE INVENTION

Iodinated contrast agents are routinely used in diagnostic and interventional medical procedures to assist in the visualization of body organs and the structures around them. The chemical structure of these agents includes one or more iodine atoms, which imparts the necessary opaqueness towards X-rays. They are most often administered intravenously but can be administered intraarterially, intrathecally, orally and intraabdominally. They are usually safe and adverse effects are generally mild and self-limiting. Nonetheless, severe or life-threatening reactions and complications can occur.

Contrast media toxicity and adverse effects can result from the chemotoxicity of the contrast agent and/or its degradants, the osmolality of the contrast medium, and the ionic composition (or lack thereof) of the contrast medium. In coronary angiography, for example, injection of contrast media into the circulatory system has been associated with several serious effects on cardiac function. In this procedure the contrast medium, rather than blood, flows through the circulatory system for a brief period of time. Due to the differences in ionic composition between blood and the contrast medium, undesirable effects can be observed such as arrhythmias, QT-prolongation, reduction in cardiac contractile force, and occurrence of ventricular fibrillation.

The occurrence and severity of adverse reactions related to high osmolality and ionic content has been reduced with the discovery and use of nonionic contrast agents. However research into ways to further reduce the potential for adverse reactions continues. The two main approaches have been to form dimeric structures of the contrast agents to maintain the iodine content while reducing the osmolality, and to add small amounts of physiologic salts to the formulations.

Another of the potential toxicities of iodinated contrast agents results from the release of iodine following degradation. The released iodine species such as molecular iodine, I₂, and iodide ion, I⁻, are thought to be causative agents in toxicity to the cells of the kidney (Sendeski, M. Clin Exp Pharmacol Physiol (2011) 38: 292-299), a condition known as contrast induced nephropathy or CIN. Gastaldo et al., (J Synchrotron Radiat (2011), 18(Pt3): 456-463) reported that iodide causes toxicity in cultured endothelial HMEC cells. The toxicity was observed after incubating the cells with sodium or potassium iodide, or with the photolysis products generated by irradiating an iodinated contrast agent with low energy X-rays. Joubert, et al., (Int J Radiation Oncology Biol Phys (2005), 62(5): 1486-1496) reported that X-ray irradiation of the iodinated contrast agent iomeprol produced iodide and other degradants, and the irradiated contrast agent was toxic to bovine aortic endothelial cells while the non-irradiated contrast agent was not toxic.

Iodine can also elicit an allergic response. Shionoya, et al., (J Tax Sci (2004), 29(2): 137-145) reported the occurrence of allergic response in guinea pigs dosed with iodinated proteins. They also demonstrated the formation of iodine and iodide ions in solutions containing ionic (iothalamate sodium) and non-ionic (iohexol) contrast media after exposure to ultraviolet light, and that the iodine was then capable of iodinating proteins.

Degradation of contrast media with resultant formation of iodine and iodide species can also result from heat exposure such as during heat sterilization, i.e. thermal degradation, and from exposure to visible and ultraviolet light, i.e. photodegradation (Eloy, et al., Clin Mater (1991), 7: 89-197).

Cyclodextrins and their derivatives are widely used in liquid formulations to enhance the aqueous solubility of hydrophobic compounds by forming inclusion complexes. Their presence in formulations can also increase, decrease, or have no effect on photodegradation (Glass, et al., Int J Photoenergy, (2001), 3: 205-211).

The inventor has identified improved formulations containing iodinated contrast agents and substituted cyclodextrins that demonstrate reduced chemical degradation when exposed to ultraviolet or visible light. The formulations are biocompatible and can be rapidly administered into the vessels of the heart with little or no alterations of cardiac function. The formulations can also be sterilized by heat without significant chemical degradation.

### SUMMARY OF THE INVENTION

The present invention encompasses iodinated contrast agent compositions with improved stabilization against chemical degradation caused by exposure of the compositions to visible or ultraviolet light. The invention provides aqueous pharmaceutical compositions having a pH of 5 to 8 and comprising an iodinated contrast agent or a salt thereof, a pharmaceutically acceptable buffering agent, and a substituted cyclodextrin present at a contrast agent to substituted cyclodextrin mole ratio from 1:0.01 to 1:2. These formulations exhibit less chemical degradation, e.g. less formation of iodine species, upon exposure to ultraviolet or visible light as compared to a corresponding composition which does not contain a substituted cyclodextrin.

The present invention encompasses ready to use, sterile, injectable, aqueous pharmaceutical compositions having a pH of 5 to 8 and comprising an iodinated contrast agent and a substituted cyclodextrin present at a contrast agent to substituted cyclodextrin mole ratio of 1:0.01 to 1:0.1. These formulations include iodinated contrast agents such as, for example, iohexol, iopamidol, iodixanol, ioversol, iopromide and ioxaglate. In certain embodiments, the formulation includes 1 to 4 mg/ml tromethamine (TRIS) buffer, or 0.1 to 0.6 mg/ml disodium calcium edetate, or both 1 to 4 mg/ml TRIS buffer and 0.1 to 0.6 mg/ml disodium calcium edetate. The substituted cyclodextrin includes sulfoalkyl ether cyclodextrins, e.g., a sulfobutylether beta cyclodextrin, and hydroxyalkyl ether cyclodextrins, e.g., a 2-hydroxypropyl beta cyclodextrin. In certain embodiments, the formulation is packaged in a primary container which does not possess enhanced light shielding properties. In other embodiments, the formulation is heat sterilized after it is packaged in the primary container.

### BRIEF DESCRIPTION OF THE DRAWINGS

FIG. 1 illustrates the binding constant of iohexol with sulfobutylether β-cyclodextrin at various cyclodextrin:iohexol mole ratios. Error is standard error of the mean.

FIG. 2 shows the change in cardiac QTc interval in dogs receiving multiple doses of iohexol (□) or iohexol plus sulfobutylether beta-cyclodextrin (▪) injected into the left coronary artery. Error is standard deviation with n=3.

FIG. 3 shows the change in left ventricular contractility, LV dP/dTₘₐₓ, in dogs receiving multiple doses of iohexol (□) or iohexol plus sulfobutylether beta-cyclodextrin (▪) injected into the left coronary artery. Error is standard deviation with n=3.

### DETAILED DESCRIPTION OF THE INVENTION

Unless otherwise specified, all percentages and amounts expressed herein and elsewhere in the specification should be understood to refer to percent by weight. The concentration is denoted in mg/mL. Also, the term “about,” when used in reference to a range of values, should be understood to refer to either value in the range, or to both values in the range.

As used throughout, ranges are used as shorthand for describing each and every value that is within the range. Any value within the range can be selected as the terminus of the range.

All documents, for example, scientific publications, patents, patent applications and patent publications, recited herein are hereby incorporated by reference in their entirety to the same extent as if each individual document was specifically and individually indicated to be incorporated by reference in its entirety. In the event of a conflict in a definition in the present disclosure and that of a cited reference, the present disclosure controls.

### Definitions

The indefinite articles “a” and “an,” as used herein in the specification and in the claims, unless clearly indicated to the contrary, should be understood to mean “at least one.”

As used herein, “or” is understood to mean inclusively or, i.e., the inclusion of at least one, but including more than one, of a number or list of elements. Only terms clearly indicated to the contrary, such as “exclusively” or “exactly one of,” will refer to the inclusion of exactly one element of a number or list of elements.

As used herein, the term “acidifying agent” is intended to mean a compound used to provide an acidic medium. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “alkalizing agent” is intended to mean a compound used to provide an alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

The terms “alkylene” and “alkyl,” as used herein (e.g., in the —O—(C₂-C₆-alkylene) SO₃ group or in the alkylamines), include linear, cyclic, and branched, saturated and unsaturated (i.e., containing one double bond) divalent alkylene groups and monovalent alkyl groups, respectively. The term “alkanol” in this text likewise includes linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

As used herein, the term “antioxidant” is intended to mean an agent that inhibits oxidation and thus is used to prevent the deterioration of preparations by the oxidative process. Such compounds include, by way of example and without limitation, acetone, potassium metabisulfite, potassium sulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid and sodium metabisulfite and others known to those of ordinary skill in the art.

As used herein, the term “biocompatible” refers to formulations that do not produce a toxic, injurious, or immunological response to living tissue or to compounds that produce only an insubstantial toxic, injurious, or immunological response. The heart and coronary arteries are particularly susceptible to injury from injection of large amounts of solutions that have ionic compositions different than the blood they are displacing. Several iodinated contrast agent formulations have been made more biocompatible through the addition of small amounts of sodium and/or calcium ions.

As used herein, the term “buffering agent” is intended to mean a compound used to resist change in pH upon storage, dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, tromethamine and its salts, meglumine, glycine, potassium metaphosphate, potassium phosphate, sodium bicarbonate, sodium tartrate and sodium citrate anhydrous and dihydrate and others known to those of ordinary skill in the art.

As used herein the term “chelating agent” refers to organic compounds which complex or sequester metal ions and reduce their potential to interact in drug degradation pathways such as those involving free radicals or oxidation-reduction. Such compounds include, by way of example and without limitation, ethylenediaminetetraacetic acid (EDTA, edetate), citric acid, fumaric acid, malic acid, pentetic acid, and/or salts thereof, maltol, and others known to those of ordinary skill in the art. Preferred chelating agents include citric acid and/or salts thereof, and the disodium, trisodium, tetrasodium, and disodium calcium salts of EDTA.

By “complexed” is meant “being part of a clathrate or inclusion complex with”, i.e., a complexed contrast agent is part of a clathrate or inclusion complex with a substituted cyclodextrin. Cyclodextrins are cone-shaped cyclic carbohydrates containing 6, 7, or 8 glucopyranose units. The interior cavity of the cyclodextrin structure is hydrophobic and provides a haven for hydrophobic compounds, which can fit part or all of their structure into these cavities, forming inclusion complexes. This inclusion complexation only occurs if there is sufficient enthalpic or entropic energetics to drive the inclusion (Brewster, M E and Loftsson T, Cyclodextrins as pharmaceutical solubilizers, Advanced Drug Delivery Reviews, 59 (2007) 645-666). In addition, the geometry must allow for at least partial insertion of the compound into the cyclodextrin cavity. Agents soluble in water, such as iodinated contrast agents, will typically interact poorly or not at all with the hydrophobic cavities of cyclodextrins, and form no inclusion complexes.

The actual percent of a compound that is complexed will vary according to the complexation equilibrium constant characterizing the complexation of a specific substituted cyclodextrin to a specific compound and to the concentrations of the substituted cyclodextrin and compound available for complexation. The complexation constant between a cyclodextrin and an insoluble agent can be determined experimentally by conducting phase solubility studies (Higuchi, T. and Connors, K. A. in “Advances in Analytical Chemistry and Instrumentation Vol. 4” Reilly, Charles N. Ed., John Wiley & Sons., 1965, pp. 117-212) where the solubility of a drug is determined in the presence of increasing amounts of a cyclodextrin or substituted cyclodextrin. When the agent is water soluble, as is the case with the iodinated contrast agents, an alternate approach must be used such as the membrane permeation method described by Ono, et al. (Eur. J. Pharm. Sci., 8 (1999) 133-139) or variations thereof.

As used herein, the transitional phrases “comprising”, “consisting essentially of” and “consisting of” define the scope of the appended claims with respect to what un-recited additional components, if any, are excluded from the scope of the claim. The term “comprising” is intended to be inclusive or open-ended and does not exclude additional, un-recited elements or method steps. The phrase “consisting of” excludes any element, step, or ingredient not specified in the claim. The phrase “consisting essentially of” limits the scope of a claim to the specified materials or steps and those that do not materially affect the basic and novel characteristic(s) of the claimed invention. All compositions or formulations identified herein can, in alternate embodiments, be more specifically defined by any of the transitional phrases “comprising”, “consisting essentially of” and “consisting of,” although, for the sake of brevity, generally “comprising” is utilized in the compositions described herein.

As used herein the term “cyclodextrin” or “CD” refers to compounds encompassed by the formula 1:

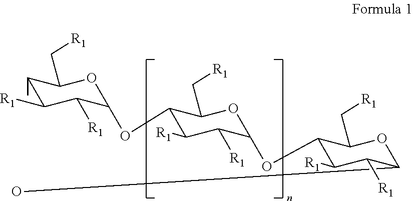

wherein n is 4, 5 or 6 and R₁ is at each occurrence —OH. The terms alpha-cyclodextrin, beta-cyclodextrin and gamma-cyclodextrin refer to cyclodextrins wherein n is 4, 5 and 6 respectively. The term “substituted cyclodextrin” or “SCD” refers to a cyclodextrin of the formula 1 wherein R₁ is selected at each occurrence from —OH or a different chemical substituent and at least one R₁ is the different chemical substituent. The substituted cyclodextrin can contain a single type of chemical substituent or more than one type within the same cyclodextrin molecule. For example, a cyclodextrin can have one —OH group substituted with a sulfoalkyl ether substituent and another —OH group substituted with a hydroxyalkyl ether substituent. Substituted cyclodextrin compounds include, by way of example and without limitation, sulfoalkyl ether cyclodextrins, hydroxyalkyl ether cyclodextrins, sulfoalkyl ether-hydroxyalkyl ether cyclodextrins, sulfoalkylether-alkyl ether cyclodextrins, alkylether cyclodextrins, hydroxybutenyl ether derivatives, hydroxybutenyl sulfonate or sulfinate derivatives and mixtures thereof, carboxyalkyl thio derivatives, and others known to those of ordinary skill in the art.

The number of hydroxyl groups in a cyclodextrin that have been replaced by a different chemical substituent is represented by a number referred to as the degree of substitution (DS). It should be noted that preparation of substituted cyclodextrins occurs in a controlled, although not exact manner. For this reason, the degree of substitution is actually a number representing the average number of substituent groups per cyclodextrin. For example, SBE7-β-CD, has an average of about 7 sulfobutylether substitutions per beta (β) cyclodextrin and HP4-β-CD has an average of about 4 hydroxypropyl substitutions. In addition, the regiochemistry of substitution of the hydroxyl groups of the cyclodextrin is variable with regard to the substitution of specific hydroxyl groups of each hexose ring. For this reason, substitution of different hydroxyl groups is likely to occur during manufacture of the substituted cyclodextrin, and a particular substituted cyclodextrin will possess a preferential, although not exclusive or specific, substitution pattern.

As used herein the term “sulfoalkyl ether cyclodextrin” or “SAE-CD” refers to compounds encompassed by the formula 1 wherein: n is 4, 5 or 6; R₁ is independently selected at each occurrence from —OH or a —O(C₂-C₆ alkylene)SO₃⁻Y⁺ group; and at least one R₁ is independently a —O(C₂-C₆ alkylene)SO₃⁻Y⁺ group, preferably a —O(CH₂)ₘSO₃⁻Y⁺ group, wherein m is 2 to 6, preferably 2 to 4, (e.g. —OCH₂CH₂CH₂SO₃⁻Y⁺ or —OCH₂CH₂CH₂CH₂SO₃⁻Y⁺) and Y⁺ is independently selected at each occurrence from the group consisting of pharmaceutically acceptable cations. In certain illustrative embodiments, n is 5; R₁ is at each occurrence —OH or —O((CH₂)₄)SO₃⁻Na⁺ and at least one R₁ is independently —O((CH₂)₄)SO₃⁻Na⁺. In certain embodiments, the SAE-CD is represented by formula 2:

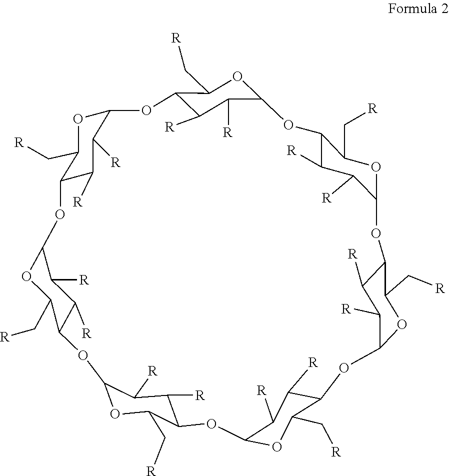

wherein R=(OH)₂₁₋ₙ or (OCH₂CH₂CH₂CH₂SO₂ONa)ₙ and where n=6 to 7. In certain illustrative embodiments, the sulfoalkyl ether cyclodextrin (SAE-CD) is sulfobutyl ether 7-beta-cyclodextrin (SBE7-β-CD).

As used herein the term “hydroxyalkyl ether cyclodextrin” or “HAE-CD” refers to compounds encompassed by the formula 1 wherein: n is 4, 5 or 6; R₁ is independently selected at each occurrence from —OH or a —O(C₂-C₆ alkylene) group further substituted with at least one —OH; and wherein at least one R1 is independently a —O(C₂-C₆ alkylene) group further substituted with at least one —OH. In certain illustrative embodiments, n is 5; R₁ is at each occurrence —OH or —OCH₂CH(OH)CH₃; and at least one R₁ is independently —OCH₂CH(OH)CH₃. In certain embodiments, the HAE-CD is represented by formula 2 wherein R=(OH₂₁₋ₙ or (OCH₂CHOHCH₃)ₙ and where n=4 to 6. In certain illustrative embodiments, the HAE-CD is 2-hydroxypropyl-4-beta-cyclodextrin. In certain other illustrative embodiments, the HAE-CD is 2-hydroxypropyl-6-beta-cyclodextrin.

As used herein the term “sulfoalkyl ether-hydroxyalkyl ether cyclodextrin” or “SAE-HAE-CD” refers to compounds encompassed by the formula 1, wherein: n is 4, 5 or 6; R₁ is independently selected at each occurrence from —OH, —O(C₂-C₆ alkylene)SO₃⁻Y⁺ wherein y+ is a pharmaceutically acceptable cation, or a —O(C₂-C₆ alkylene) group further substituted with at least one —OH; at least one R1 is independently —O(C₂-C₆ alkylene)SO₃⁻Y⁺ wherein Y⁺ is a pharmaceutically acceptable cation; and at least one R₁ is independently a —O(C₂-C₆ alkylene) group further substituted with at least one —OH. In certain illustrative embodiments, n is 5; R₁ is at each occurrence —OH, —O((CH₂)₄)SO₃⁻Na⁺, or —OCH₂CH(OH)CH₃; at least one R₁ is independently —O((CH₂)₄)SO₃⁻Na⁺ and at least one R₁ is independently —OCH₂CH(OH)CH₃. In certain embodiments, the SAE-HAE-CD is represented by formula 2 wherein R=(OH₂₁₋ₙ₋ₚ or (OCH₂CH₂CH₂CH₂SO₂ONa)ₙ or (OCH₂CH(OH)CH₃)ₚ and where n=2 to 6 and p=1 to 6, more preferably where n=3 or 4 and p=3 or 4.

As used herein the term “sulfoalkyl ether-alkyl ether cyclodextrin” or “SAE-AE-CD” refers to compounds encompassed by the formula 1, wherein: n is 4, 5 or 6; R₁ is independently selected at each occurrence from —OH, —O(C₂-C₆ alkylene)SO₃⁻Y⁺ wherein Y⁺ is a pharmaceutically acceptable cation, or a —O(C₂-C₆ alkylene) group; at least one R₁ is independently —O(C₂-C₆ alkylene)SO₃⁻Y⁺ wherein Y⁺ is a pharmaceutically acceptable cation; and at least one R₁ is independently a —O(C₂-C₆ alkylene) group. In certain illustrative embodiments, n is 5; R₁ is at each occurrence —OH, —O((CH₂)₄)SO₃⁻Na⁺, or —OCH₂CH₃; at least one R₁ is independently —O((CH₂)₄)SO₃⁻Na⁺; and at least one R₁ is independently —OCH₂CH₃. In certain embodiments, the SAE-AE-CD is represented by formula 2 wherein R=(OH₂₁₋ₙ₋ₚ or (OCH₂CH₂CH₂CH₂SO₂ONa)ₙ or (OCH₂CH₃)ₚ and where n=4 or 6 and p=4 or 6. In certain illustrative embodiments, the SAE-AE-CD is sulfobutylether 3.5-ethylether 3.5-beta-cyclodextrin (SBE3.5-EE3.5-β-CD). In certain other illustrative embodiments, the SAE-AE-CD is sulfobutylether 4-ethylether 4-beta-cyclodextrin (SBE4-EE4-β-CD). Sulfoalkyl ether-alkyl ether cyclodextrins are disclosed in U.S. Pat. No. 7,625,878

As used herein the term “alkyl ether cyclodextrin” or “AE-CD” refers to compounds encompassed by the formula 1 wherein: n is 4, 5 or 6; R₁ is independently selected at each occurrence from —OH or a —O(C₁-C₆ alkylene) group; and wherein at least one R₁ is independently a —O(C₁-C₆ alkylene) group. In certain illustrative embodiments, R₁ is at each occurrence —OH or —OCH₃; at least one R₁ is independently —OCH₃; and at least one R₁ is independently-OH. These alkyl ether cyclodextrins are referred to as “partially methylated” cyclodextrins. In certain illustrative embodiments, n is 5; R₁ is at each occurrence —OH or —OCH₃; and at least one R₁ is independently —OCH₃. In certain embodiments, the AE-CD is represented by formula 2 wherein R=(OH₂₁₋ₙ or (OCH₃)ₙ and where n=4. In certain illustrative embodiments, the AE-CD is methyl 4-beta-cyclodextrin (Me4-β-CD).

As used herein the term “hydroxybutenyl ether cyclodextrin” or “HBen-CD” refers to compounds encompassed by the formula 1 wherein: n is 4, 5 or 6; R1 is independently selected at each occurrence from —OH, —OCH₂CH(OX)CHCH₂, —OCH(CHCH₂)CH₂OX, —OCH₂CH(OX)CH₂CH₂SO₃M, —OCH₂CH(OX)CH(SO₂M)CH₂SO₃M, —OCH(CH₂OX)CH₂CH₂SO₃M, or —OCH(CH₂OX)CH(SO₂M)CH₂SO₃M where X is H or another hydroxybutenyl group and M is a pharmaceutically acceptable cation; and wherein at least one R₁ is independently —OCH₂CH(OX)CHCH₂, —OCH(CHCH₂)CH₂OX, —OCH₂CH(OX)CH₂CH₂SO₃⁻M, —OCH₂CH(OX)CH(SO₂M)CH₂SO₃M, —OCH(CH₂OX)CH₂CH₂SO₃M, or —OCH(CH₂OX)CH(SO₂M)CH₂SO₃M where X is H or another hydroxybutenyl group and M is a pharmaceutically acceptable cation. Hydroxybutenyl ether cyclodextrins are disclosed in U.S. Pat. Nos. 6,479,467 and 6,610,671.

As used herein the term “carboxyalkyl thio cyclodextrin” or “CAT-CD” refers to compounds encompassed by the formula 1 wherein: n is 4, 5 or 6; R₁ is independently selected at each occurrence from —OH or a —S(CH₂)_{z}CO₂M group where Z is 1-4, and M is a pharmaceutically acceptable cation; and wherein at least one R₁ is independently a —S(CH₂)_{z}CO₂M group where Z is 1-4 and M is a pharmaceutically acceptable cation. In certain embodiments n is 6 and eight of the R₁ groups are —SCH₂CH₂CO₂M, where M is sodium. In certain embodiments, the CAT-CD is Sugammadex™ (trade name Bridion). Carboxyalkyl thio cyclodextrins are disclosed in U.S. Pat. No. 6,949,527.

The liquid formulation of the invention will comprise an effective amount of an iodinated contrast agent or a salt thereof. An effective amount of an iodinated contrast agent, or salt thereof, is an amount or quantity of the iodinated contrast agent, or salt thereof, that will block X-rays and provide a visual contrast between the agent and surrounding tissue when administered to a subject undergoing an X-ray procedure.

As used herein the term “heat sterilization” refers to the process of exposing materials to elevated temperatures for sufficient time and temperature to kill or inactivate any microorganisms present to a level acceptable for the intended use of the materials. For pharmaceutically acceptable solutions intended for parenteral administration, the generally acceptable level is a 10-6 microbial survivor probability, meaning there is less than one chance in 1 million that viable microorganisms are present. The heat sterilization can be conducted with dry heat in a chamber expressly designed for that purpose, or by steam sterilization in a chamber called an autoclave. Steam sterilization is typically conducted at a temperature of about 121-123° C. for periods of 15 to 30 minutes, though other temperatures and durations may be used, for example 115-116° C. for at least 30 minutes, 126-129° C. for at least 10 minutes or 134-138° C. for at least 3 minutes. Dry heat sterilization typically requires higher temperatures and exposure times than steam heat sterilization, for example 160° C. for at least 180 minutes, 170° C. for at least 60 minutes or 180° C. for at least 30 minutes. Other temperatures and times can be used for both steam sterilization and dry heat sterilization as known by those skilled in the art. Materials which have undergone the process of heat sterilization are said to be “heat sterilized”. In general, compositions of the invention are heat sterilized after packaging into a primary container. Requirements for sterilization of solutions and methods for evaluating sterility can be found in various pharmacopeial compendia of standards such as the United States Pharmacopeia, the Japanese Pharmacopoeia, the European Pharmacopoeia, the International Pharmacopeia, the British Pharmacopoeia, the Indian Pharmacopoeia, the Pharmacopoeia of the People's Republic of China, and others.

As used herein the term “inert gas” refers to a gas normally used to provide an inert atmosphere in containers containing pharmaceutical compositions. The gas is added to the containers to displace oxygen that is present and prevent the oxygen from facilitating degradation of the composition. When the composition is a liquid, the gas is also sometimes passed through the liquid to displace dissolved oxygen. Inert gasses include, by way of example and without limitation, argon, nitrogen, helium, carbon dioxide and others known to those of ordinary skill in the art.

As used herein the term “iodinated contrast agent” refers to iodine containing organic compounds used to assist in the visualization of body organs and the structures around them in diagnostic and interventional medical procedures using X-rays. The iodine present in the compounds blocks part of the X-rays and thus provides a contrasting visualization to the surroundings not containing the contrast. The chemical structure of iodinated contrast agents contains a benzene ring substituted with 1, 2, or 3 iodine atoms. Various additional chemical groups are substituted onto the benzene ring to impart desired properties such as increased water solubility. These groups can be ionic or neutral in charge and two of the iodinated benzene rings can be attached together with various chemical linking groups to form dimeric compounds. Dimeric iodinated contrast agents can be advantageous since solutions containing them can contain the same amount of iodine as a corresponding monomeric solution but have a lower osmolality.

The iodinated contrast agents can be divided into four groups; ionic monomers, ionic dimers, nonionic monomers, and nonionic dimers. Examples of ionic monomers include but are not limited to, acetrizoic acid, diatrizoic acid, iodamic acid, ioglicic acid, iopanoic acid, iopronic acid, iotalamic acid, ioxitalamic acid, ipodic acid, metrizoic acid, and/or salts thereof. Examples of ionic dimers include but are not limited to, iocarmic acid, iodipamide, iodoxamic acid, ioxaglic acid, and/or salts thereof. Examples of nonionic monomers include but are not limited to, iobitridol, iohexol, iomeprol, iopamidol, iopentol, iopromide, iosimide, ioversol, ioxilan, and metrizamide. Examples of nonionic dimers include but are not limited to, iodixanol, ioforminol, and iotrolan.

In one embodiment of any of the compositions described herein, the iodinated contrast agent is selected from the group consisting of: the ionic agents iocarmic acid, iodipamide, iodoxamic acid, ioxaglic acid, acetrizoic acid, diatrizoic acid, iodamic acid, ioglicic acid, iopanoic acid, iopronic acid, iothalamic acid, ioxitalamic acid, ipodic acid, metrizoic acid, and their pharmaceutically acceptable salts, and the nonionic agents iodixanol, ioforminol, iotrolan, iobitridol, iohexol, iomeprol, iopamidol, iopentol, iopromide, iosimide, ioversol, ioxilan, and metrizamide.

The iodinated contrast agent iopamidol is sold under the names Iopamiro®, Isovue®, Iopamiron™ and Niopam™ Iodixanol is sold under the name Visipaque®. Ioversol is sold under the name Optiray™. Iopromide is sold under the name Ultravist®. Ioxaglate is sold under the name Hexabrix® (which contains ioxaglate meglumine and ioxaglate sodium). Iohexol is sold under the name Omnipaque®.

As used herein the term “iodine content” refers to the amount of organically bound iodine contained in the chemical structure of an iodinated contrast agent or in a formulation of an iodinated contrast agent. It is commonly reported as the percentage by weight of the contrast agent, or the weight per volume concentration in a solution comprising the contrast agent. For example, iohexol has a molecular weight of 821.14 g/mole and contains 3 iodine atoms of molecular weight 126.9 g/mole in each molecule. Its iodine content is 46.36%. An iohexol solution formulation comprising 755 mg/mL iohexol contains 350 mg iodine per milliliter or 350 mgl/mL. The iodine content of contrast agent formulations commonly used in X-ray procedures ranges from 140 to 400 mgl/mL.

As used herein, the term “iodine species” refers to those forms of iodine that are formed by degradation of an iodinated contrast agent by photolysis (e.g. after irradiation by ultraviolet or visible light) or by exposure to thermal stress such as by autoclaving. These iodine species are no longer organically bound to the contrast agent's chemical structure. Exemplary iodine species include iodide (r), triiodide (h−), iodate (10-3), and elemental iodine (h). The iodine species are in equilibrium with each other and can interconvert depending on the pH of the medium. Analysis of each of the individual species is possible, but the easiest analysis is to convert all the species to r through the addition of an excess of sodium thiosulfate. The r can then be measured by chromatographic separation with ultraviolet detection at 230 nm.

As used herein the term “pH adjusting agent” is an agent to increase or decrease the desired pH of the formulation when admixed into the formulation. The pH of the liquid formulation will generally range from about pH 5.5 to about pH 8.0; however, liquid formulations having higher or lower pH values can also be prepared. It is contemplated that the iodinated contrast agent chemical stability can be increased by optimizing the pH as well as the mole ratio of substituted cyclodextrin to contrast agent. The pH of the composition may be adjusted using an appropriate pH adjusting agent, such as a suitable acid, base, amine, or any combination thereof. Preferably, a pH adjusting agent used in the formulation include hydrochloric acid, sodium hydroxide, amines, ammonium hydroxide, nitric acid, phosphoric acid, sulfuric acid, citric acid, organic acids, and/or salts thereof, and any combination thereof.

As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, a “pharmaceutically acceptable liquid carrier” is any aqueous medium used in the pharmaceutical sciences for dilution or dissolution of parenteral formulations. In a specific embodiment, the term “pharmaceutically acceptable” means generally accepted by or approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a formulation of the invention is administered. Such pharmaceutical carriers can be liquids, such as water, saline, aqueous solutions and the like. When administered to a patient, the formulations of the invention and pharmaceutically acceptable vehicles are preferably sterile. Water is a preferred vehicle when the compound of the invention is administered intravenously or intraarterially.

As used herein, the term “pharmaceutically acceptable cation” is intended to mean a cation selected from the group comprising H⁺, alkali metals (e.g., Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine, ethylenediamine and (C₄-C₈)-cycloalkanolamine, and other cations known to be pharmaceutically acceptable to those skilled in the art.

As used herein, the term “pharmaceutically acceptable container” is intended to mean a container closure system that: protects the drug product, for example, from factors that can cause degradation of the dosage form over its shelf-life; is compatible with the drug product, for example, the packaging components will not interact sufficiently to cause unacceptable changes in the quality of either the drug or the packaging component, such as absorption or adsorption of the drug substance, degradation of the drug substance that is induced by extractables/leachables from the container, precipitation, and changes in pH; and is safe, for example, a container that does not leach harmful or undesirable amounts of substances to which a patient will be exposed when being treated with the product, or in the case of injectable formulations, the container will protect the formulation from the introduction of microbes and not contain pyrogens. Containers useful for injectable formulations are often sterilized prior to and/or after being filled with the formulation. Pharmaceutically acceptable containers include, but are not limited to, polymer or glass bottles, vials, syringes or cartridges for autoinjectors. Containers for autoinjectors are described for example in U.S. Pat. Nos. 5,383,858, 5,997,502, 6,322,535, and 6,402,718. Suitable pharmaceutically acceptable containers include an evacuated container, a syringe, bag, pouch, ampoule, vial, bottle, or any pharmaceutically acceptable device known to those skilled in the art for the delivery of liquid formulations. To shield the compositions from light, amber colored vials or syringes can be used, and/or the packaging can further include a light barrier, such as an aluminum overpouch. Containers with light-shielding properties by nature have poor light permeability. In these containers, during production steps or during storage-related quality tests, visual or mechanical inspection for any insoluble foreign matter is difficult to perform. By the combined use of these light-shielding materials, their light-shielding properties can be further enhanced. Containers that do not possess enhanced light shielding properties, i.e., are transparent, are those containers which do not shield light from entering the container. In the field of quality control or quality tests, use of transparent vials is desirable.

As used herein, the term “primary container” is intended to mean a pharmaceutically acceptable container that has direct physical contact with the drug product or formulation. This include, for example, an evacuated container, a syringe, a bag, pouch, ampoule, vial, bottle, or any pharmaceutically acceptable device known to those skilled in the art for the delivery of liquid formulations.

As used herein, the term “photolysis” is intended to mean chemical degradation that occurs when a compound is irradiated with ultraviolet or visible light. The degradants formed by photolysis of iodinated contrast agents include, but are not limited to, iodine species.

Ready to use, injectable formulations described herein are stable, allow medical personal to use prepared containers containing an injectable formulation off the shelf without additional preparation, avoid potential contamination problems, and eliminate dosage errors. Additional benefits of premixed, ready to use, injectable pharmaceutical compositions include convenience and ease of use, improved safety for patients (due to elimination of dosage errors and solution contamination), reduction of medical waste, and ease of administration in emergency situations. Such pharmaceutical compositions described herein require no dilution prior to administration.

### Compositions of the Invention

The invention encompasses compositions indicated for intravascular administration in subjects requiring radiographic visualization. Intravascular injection of these agents specifies those vessels in the path of flow of the contrast agent, permitting radiographic visualization of the internal structures until significant dilution and elimination occurs.

The invention is based on the finding that certain compositions containing iodinated contrast agents and a substituted cyclodextrin show reduced degradation when exposed to ultraviolet or visible light.

The compositions of the invention encompasses liquid formulations including an iodinated contrast agent or a salt thereof that can be administered parenterally, for example, intravenously or intraarterially, to a subject in need thereof.

In one embodiment, the invention provides an aqueous pharmaceutical composition having a pH of 5 to 8 and comprising an iodinated contrast agent; a pharmaceutically acceptable buffering agent; and a substituted cyclodextrin present at a contrast agent to substituted cyclodextrin mole ratio from 1:0.01 to 1:2. In an embodiment, the composition will exhibit a reduction in formation of iodine species when exposed to ultraviolet (UV) light as compared to a corresponding composition, without a substituted cyclodextrin, exposed to the same ultraviolet light. In certain embodiments, the composition exhibits at least a 3% reduction to at least a 60% reduction of iodine species. In certain embodiments, the composition exhibits at least a 3% at least a 5%, at least a 10%, at least a 20%, at least a 30%, at least a 40%, at least a 50%, or at least a 60%, reduction in formation of iodine species as compared to the corresponding composition.

In another embodiment, the invention provides an aqueous pharmaceutical composition having a pH of 5 to 8 and comprising an iodinated contrast agent selected from the group consisting of iohexol, iopromide, ioversol, ioxaglate, and iodixanol; a pharmaceutically acceptable buffering agent; and a substituted cyclodextrin present at a contrast agent to substituted cyclodextrin mole ratio from 1:0.01 to 1:2. In an embodiment, the composition will exhibit a reduction in formation of iodine species when exposed to visible light as compared to a corresponding composition, without a substituted cyclodextrin, exposed to the same visible light. In certain embodiments, the composition exhibits at least a 3% reduction to at least a 60% reduction of iodine species. In certain embodiments, the composition exhibits at least a 3% at least a 5%, at least a 10%, at least a 20%, at least a 30%, at least a 40%, at least a 50%, or at least a 60%, reduction in formation of iodine species as compared to the corresponding composition.

In one embodiment, the iodinated contrast agent is any one of the iodinated contrast agents described herein, including but not limited to the ionic agents iocarmic acid, iodipamide, iodoxamic acid, ioxaglic acid, acetrizoic acid, diatrizoic acid, iodamic acid, ioglicic acid, iopanoic acid, iopronic acid, iothalamic acid, ioxitalamic acid, ipodic acid, metrizoic acid, and their pharmaceutically acceptable salts, and the nonionic agents iodixanol, ioforminol, iotrolan, iobitridol, iohexol, iomeprol, iopamidol, iopentol, iopromide, iosimide, ioversol, ioxilan, and metrizamide.

In one embodiment, the iodinated contrast agent is selected from the group consisting of iopamidol, iodixanol, iopromide and ioxaglate. In another embodiment, the iodinated contrast agent is selected from the groups consisting of iohexol, ioversol, diatrizoate meglumine and ioxaglate and the substituted cyclodextrin is selected from the groups consisting of sulfoalkylether cyclodextrins, partially methylated cyclodextrins, sulfoalkylether alkylether cyclodextrins and sulfoalkyl ether hydroxyalkyl ether cyclodextrins.

In one embodiment, the substituted cyclodextrin is any one of the cyclodextrins described herein. In another embodiment, the substituted cyclodextrin is selected from the groups consisting of sulfoalkylether cyclodextrins, 2-hydroxypropyl cyclodextrins, partially methylated cyclodextrins and sulfoalkylether alkylether cyclodextrins. In a further embodiment, the substituted cyclodextrin is selected from a group consisting of a sulfobutyl ether beta-cyclodextrin, a sulfobutyl ether gamma-cyclodextrin, a sulfobutyl ether alpha-cyclodextrin, a sulfopropyl ether beta-cyclodextrin, a sulfobutylether ethylether beta-cyclodextrin, a 2-hydroxypropyl beta cyclodextrin, and a partially methylated beta cyclodextrin. In one embodiment, the substituted cyclodextrin is a sulfobutyl ether beta cyclodextrin with an average degree of substitution of 7, i.e. SBE-7-βCD. In another embodiment, the substituted cyclodextrin is a 2-hydroxypropyl beta cyclodextrin with an average degree of substitution of 6.3 or 4.3, e.g., HP-6.3-βCD or HP-4.3-βCD.

In one embodiment, the UV light exposure is 119 watt hours per square meter. In another embodiment, the UV light exposure is 159 watt hours per square meter. In one embodiment, the visible light exposure is 0.65 million lux hours. In another embodiment, the visible light exposure if 0.52 million lux hours.

In certain embodiments, the composition is heat sterilized prior to exposure to the UV or visible light. In one embodiment, the heat sterilization comprises steam sterilization. In another embodiment, the steam sterilization occurs at 115-116° C. for at least 30 minutes, 121-123° C. for at least 15 minutes, 126-129° C. for at least 10 minutes, or 134-138° C. for at least 3 minutes. In other embodiments, the composition is dry heat sterilized prior to exposure to the UV or visible light. In one embodiment, the dry heat sterilization occurs at 160° C. for at least 180 minutes, 170° C. for at least 60 minutes or 180° C. for at least 30 minutes. In another embodiment, the heat sterilization is conducted at a sufficient temperature and for a sufficient time to assure at least a 10⁻⁶ microbial survivor probability.

In one embodiment, the pharmaceutically acceptable buffering agent is any of the buffering agents described herein. In another embodiment the pharmaceutically acceptable buffering agent is selected from the group consisting of tromethamine (TRIS), phosphate, and meglumine, and their pharmaceutically acceptable salts. In one embodiment, the composition has a pH of 6.5 to 7.7. In another embodiment, the composition has a pH of 7.4. In another embodiment, the composition further comprises one or more components selected from the group consisting of pH adjusting agents, antioxidants, chelating agents, and inert gasses. Examples of such components are described elsewhere herein.

In one embodiment, the composition has an iodine content of about 19 mgl/mL to about 400 mgl/mL. In another embodiment, the iodine content is 111 mgl/mL to about 400 mgl/mL. In another embodiment, the iodine content is greater than 150 mgl/mL and less than or equal to 400 mgl/mL.

In one embodiment, the contrast agent to substituted cyclodextrin mole ratio is from about 1:0.01 to about 1:2. In another embodiment, the contrast agent to substituted cyclodextrin mole ratio is from about 1:0.02 to about 1:2. In another embodiment, the contrast agent to substituted cyclodextrin mole ratio is from greater than about 1:0.025 to 1:2. In one embodiment, the contrast agent to substituted cyclodextrin mole ratio is from about 1:0.01 to about 1:0.1. In another embodiment, the contrast agent to substituted cyclodextrin mole ratio is from about 1:0.02 to about 1:0.1. In another embodiment, the contrast agent to substituted cyclodextrin mole ratio is from greater than about 1:0.025 to 1:0.1.

In one embodiment, the invention provides a ready to use, sterile, injectable aqueous pharmaceutical composition have a pH of 5 to 8 and comprising an iodinated contrast selected from the group consisting of iohexol, iopamidol, iodixanol, ioversol, and iopromide; 1 to 4 mg/ml tromethamine (TRIS) buffer, 0.1 to 0.6 mg/mL disodium calcium edetate; and a substituted cyclodextrin selected from the group consisting of sulfobutylether beta cyclodextrins and 2 hydroxypropyl beta cyclodextrins, wherein the substituted cyclodextrin is present at a contrast agent to substituted cyclodextrin mole ratio from about 1:0.01 to 1:0.1. In one embodiment, the composition is packaged in a primary container that does not possess enhanced light shielding properties.

In one embodiment, the composition is heat sterilized after it is packaged in the primary container. In another embodiment, the heat sterilization comprises steam sterilization or dry heat sterilization. In one embodiment, the steam sterilization occurs at 115-116° C. for at least 30 minutes, 121-123° C. for at least 15 minutes, 126-129° C. for at least 10 minutes, or 134-138° C. for at least 3 minutes. In another embodiment, the dry heat sterilization occurs at 160° C. for at least 180 minutes, 170° C. for at least 60 minutes or 180° C. for at least 30 minutes.

In one embodiment, the composition has an iodine content greater than 150 mgl/mL and less than or equal to 400 mgl/mL. In one embodiment, the pH of the composition is 6.5 to 7.7.

In one embodiment, the iodinated contrast agent is iohexol, iopamidol, ioversol or iopromide and the iodinated contrast agent is present at a molar concentration greater than 394.1 mM and less than or equal to 1051 mM. In another embodiment, the iodinated contrast agent is iodixanol, and the iodixanol is present at a molar concentration greater than 197.1 mM and less than or equal to 525.4 mM.

In one embodiment, the iodinated contrast agent is iohexol, the substituted cyclodextrin is a sulfobutylether beta cyclodextrin or a 2-hydroxypropyl beta cyclodextrin, and the composition has an iodine content greater than 150 mgl/mL and less than or equal to 400 mgl/ml. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.8 to 7.7. In another embodiment, the pH of the composition is 7.4. In another embodiment, the composition comprises 1.21 mg/ml tromethamine and 0.1 mg/ml disodium calcium edetate. In one embodiment, the iohexol to substituted cyclodextrin is 1:0.02 to 1:0. 1 or greater than 1:0.025 to 1:0.1. In another embodiment, the iodine content of the composition is 155, 180, 240, 300, 350 or 400 mgI/ml.

In one embodiment, the iodinated contrast agent is iopamidol, the substituted cyclodextrin is a sulfobutylether beta cyclodextrin or a 2-hydroxypropyl beta cyclodextrin, and the composition has an iodine content greater than 150 mgl/mL and less than or equal to 400 mgl/ml. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.5 to 7.5. In another embodiment, the pH of the composition is 7.4. In another embodiment, the composition comprises 1.0 mg/ml tromethamine and 0.26 to 0.48 mg/ml disodium calcium edetate. In one embodiment, the iopamidol to substituted cyclodextrin is 1:0.02 to 1:0. 1 or greater than 1:0.025 to 1:0.1. In another embodiment, the iodine content of the composition is 200 mgl/ml.

In one embodiment, the iodinated contrast agent is iopromide, the substituted cyclodextrin is a sulfobutylether beta cyclodextrin or a 2-hydroxypropyl beta cyclodextrin, and the composition has an iodine content greater than 150 mgl/mL and less than or equal to 400 mgl/ml. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.5 to 8.0. In another embodiment, the pH of the composition is 7.4. In another embodiment, the composition comprises 2.42 mg/ml tromethamine and 0.1 mg/ml disodium calcium edetate. In one embodiment, the iopromide to substituted cyclodextrin is 1:0.02 to 1:0.1 or greater than 1:0.025 to 1:0.1. In another embodiment, the iodine content of the composition is 340, 300 or 370 mgl/ml.

In one embodiment, the iodinated contrast agent is iodixanol, the substituted cyclodextrin is a sulfobutylether beta cyclodextrin or a 2-hydroxypropyl beta cyclodextrin, and the composition has an iodine content greater than 150 mgl/mL and less than or equal to 400 mgl/ml. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.8 to 7.7. In another embodiment, the pH of the composition is 7.4. In another embodiment, the composition comprises 1.21 mg/ml tromethamine and 0.1 mg/ml disodium calcium edetate. In another embodiment, the composition further comprises 1.11 mg/ml to 1.87 mg/ml of sodium chloride and 0.044 mg/ml to 0.074 mg/ml of calcium chloride dihydrate. In one embodiment, the iodixanol to substituted cyclodextrin is 1:0.02 to 1:0.1 or greater than 1:0.025 to 1:0.1. In another embodiment, the iodine content of the composition is 270 or 320 mgl/ml.

In one embodiment, the iodinated contrast agent is ioversol, the substituted cyclodextrin is a sulfobutylether beta cyclodextrin or a 2-hydroxypropyl beta cyclodextrin, and the composition has an iodine content greater than 150 mgl/mL and less than or equal to 400 mgl/ml. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.0 to 7.4. In another embodiment, the pH of the composition is 7.1. In another embodiment, the composition comprises 3.6 mg/ml tromethamine and 0.2 mg/ml disodium calcium edetate. In one embodiment, the ioversol to substituted cyclodextrin is 1:0.02 to 1:0.1 or greater than 1:0.025 to 1:0.1. In another embodiment, the iodine content of the composition is 160, 240, 300, 320 or 350 mgl/ml.

In another embodiment, the invention provides a ready to use, sterile, injectable aqueous pharmaceutical composition have a pH of 5 to 8 and comprising ioxaglate, wherein the ioxaglate comprises ioxaglate meglumine and ioxaglate sodium; 0.1 to 0.6 mg/mL disodium calcium edetate; and a substituted cyclodextrin selected from the group consisting of sulfobutylether beta cyclodextrins and 2 hydroxypropyl beta cyclodextrins, wherein the substituted cyclodextrin is present at a contrast agent to substituted cyclodextrin mole ratio from about 1:0.01 to 1:0.1.

In one embodiment, the composition is packaged in a primary container that does not possess enhanced light shielding properties.

In one embodiment, the composition is heat sterilized after it is packaged in the primary container. In another embodiment, the heat sterilization comprises steam sterilization or dry heat sterilization. In one embodiment, the steam sterilization occurs at 115-116° C. for at least 30 minutes, 121-123° C. for at least 15 minutes, 126-129° C. for at least 10 minutes, or 134-138° C. for at least 3 minutes. In another embodiment, the dry heat sterilization occurs at 160° C. for at least 180 minutes, 170° C. for at least 60 minutes or 180° C. for at least 30 minutes.

In one embodiment, the substituted cyclodextrin is a sulfobutylether beta cyclodextrin or a 2-hydroxypropyl beta cyclodextrin. In a further embodiment, the substituted cyclodextrin is a sulfobutylether beta cyclodextrin with an average degree of substitution of 7, i.e. SBE-7-βCD. In another embodiment, the substituted cyclodextrin is a 2-hydroxypropyl beta cyclodextrin with an average degree of substitution of 4.3 or 6.3, e.g., HP-4.3-βCD or HP-6.3-βCD. In one embodiment, the composition has a pH of 6.0 to 7.6. In another embodiment, the pH of the composition is 7.0. In one embodiment, the composition comprises 393 mg of ioxaglate meglumine and 196 mg of ioxaglate sodium. In another embodiment, the composition comprises 0.1 mg/ml disodium calcium edetate. In one embodiment, the ioxaglate to substituted cyclodextrin is 1:0.02 to 1:0.1 or greater than 1:0.025 to 1:0.1. In another embodiment, the iodine content of the composition is 320 mgl/ml.

In any of the ready to use, sterile, injectable aqueous pharmaceutical compositions described herein, the composition may further comprise one or more components selected from the group consisting of pH adjusting agents, antioxidants, chelating agents, and inert gasses.

The formulations of the inventions described herein also include water. Specific embodiments of the invention include pyrogen-free, sterile water as liquid carrier. The water can comprise other components described herein. Water suitable for injection is suitable for use in the liquid formulation of the invention.

An antioxidant may be but need not be added to the formulation of the invention. Preferred antioxidants include EDTA and salts thereof, sodium metabisulfite and pentetate, for example.

A chelating agent may be but need not be added to the formulation of the invention. Preferred chelating agents include EDTA and salts thereof, and citric acid and salts thereof.

The chemical stability of the liquid formulations of the invention can be enhanced by: adding an antioxidant, adding a chelating agent, adjusting the pH of the liquid carrier, and/or eliminating or minimizing the presence of oxygen in the formulation.

In view of the above description and the examples below, one of ordinary skill in the art will be able to practice the invention as claimed without undue experimentation. The foregoing will be better understood with reference to the following examples that detail certain compositions according to the present invention. All references made to these examples are for the purposes of illustration and not limitation. The following examples should not be considered exhaustive or exclusive, but merely illustrative of only a few of the many embodiments contemplated by the invention, as combinations of the foregoing embodiments are contemplated.

### Examples

### Example 1: Complexation of Sulfobutylether Beta-Cyclodextrin with Iohexol

The membrane permeation method for determining complexation is founded on the concept that uncomplexed agents will pass through a semi-permeable membrane whereas a cyclodextrin or an agent complexed with a cyclodextrin will not pass, if the membrane pore size is selected carefully. The permeation rate of the agent is dependent on the amounts of uncomplexed agent on either side of the membrane as a function of time. One can place cyclodextrin and agent on one side of a membrane (donor side) and measure the amount of agent crossing over time into the receptor side, and by using appropriate equations calculate the fraction of uncomplexed agent present in the donor side solution. One can then calculate the effective binding constant between the cyclodextrin and the agent.

Ono, et al. described the equations and experimental setup for the model where the permeation is allowed to continue for extended time. When the time course is limited such that the concentration of agent on the donor side does not vary significantly and that appearing on the receptor side is very small relative to the donor side, the equations collapse to a simple direct dependency (X) of permeation rate (J) on the free and uncomplexed concentration of agent on the donor side (C_{donor}) as in Equation 1.

J=X(C_{donor}) Equation 1

One can measure the permeation rate of an agent across a semi-permeable membrane in the absence or presence of a cyclodextrin, and by dividing the rate in the presence of the cyclodextrin by the rate in its absence one can determine the fraction of agent uncomplexed in the presence of that concentration of cyclodextrin. The experiment is repeated with other concentrations of cyclodextrin to obtain the fraction of uncomplexed agent at each cyclodextrin concentration.

The complexation or binding constant, K, for a 1:1 cyclodextrin:agent interaction is defined as:

$$K = \frac{ACD}{( ( {A}_{0} ) ( {CD}_{0} ) )} , \qquad Equation 2$$

where A₀ and CD₀ are the uncomplexed concentrations of agent and cyclodextrin respectively, and ACD is the concentration of the complex. The corresponding mass balance equations are:

[CD_{Total}]=[CD₀]+[ACD] Equation 3

[A_{Total}]=[A₀]+[ACD] Equation 4

where CD_{Total} and A_{Total} are the total amounts of cyclodextrin and agent respectively. Equations 2, 3, and 4 can be combined and rearranged to give an equation for CD₀:

K[CD₀]²+(K[CD_{Total}]−K[A_{Total}]+1)[CD₀]−[A_{Total}]=0 Equation 5

This equation can be solved in an iterative fashion using Newton's approximation or other methods to generate values for free cyclodextrin at fixed values for K, A_{Total}, and CD_{Total}. The corresponding values for free agent are then calculated from equations 2 and 3.

Values of Aₜₒₜₐₗ, and CDₜₒₜₐₗ used on the donor side in permeation experiments are entered into Equation 5 and then various values of K input until the calculated value of free agent on the donor side matches the value obtained in each experiment.

This procedure was used in an experiment evaluating the complexation of sulfobutylether beta-cyclodextrin with the iodinated contrast agent, iohexol. Aqueous solutions containing iohexol:cyclodextrin moles ratios of 1:0, 1:0.25, 1:0.5, 1:1, and 1:1.5 were placed on the donor side of semi-permeable cellulose ester ultrafiltration membranes (Molecular/Par®, Spectrum laboratories, molecular weight cutoff of 1000 daltons) mounted in side-by side diffusion chambers. Aqueous solutions of the cyclodextrin, adjusted to provide equal osmotic strength to the donor side, were placed on the receptor side. The permeation rates for the appearance of iohexol into the solution on the receptor side of the membrane were measured by assaying the receptor solutions periodically over time using high pressure liquid chromatography.

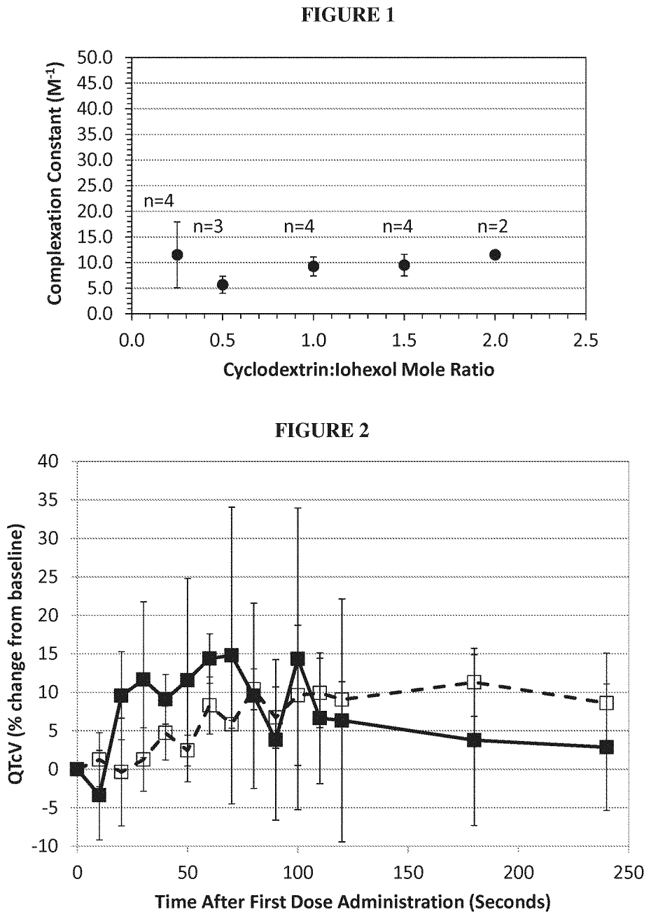

Results of the study are depicted in FIG. 1. The complexation constant is shown to be very low, averaging about 9.5 M⁻¹ indicating very low complexation. Complexation constants are typically between 50-2000 M⁻¹ (Loftsson, et al. Cyclodextrins in Drug Delivery, Expert Opin Drug Deliv, (2005) 2(2):335-351) and values lower than about 50M⁻¹ indicate little to no interaction between the cyclodextrin and agent.

### Example 2: Irradiation of Aqueous Solutions Containing Iodinated Contrast Agents

Aqueous solutions comprising iodinated contrast agents were prepared and evaluated in a photolysis chamber as described in the ICH guidance document, Q1B Photostability Testing of New Drug Substances and Products, available from the US Food and Drug Administration, incorporated herein by reference. The solutions were placed in 1 cm×1 cm (3 mL) quartz cuvettes having 2 clear sides and 2 sides etched. Lids were placed on the cuvettes and sealed with a wrap of Parafilm® around the joint. The cuvettes were placed in a photolysis chamber, upright on a flat surface beneath and centered between two horizontal 24 inch fluorescent lamps, (GE, black light F20T12, 20 watt for ultraviolet, or Philips F20T12/CW, 20 watt for visible) placed 6 cm apart. The cuvettes were positioned such that their two clear sides were facing the two lamps.

Cuvettes were spaced ˜2 cm from each other, and no closer than 15 cm from the ends of the lamps. This positioning allowed reproducible light exposure to all cuvettes.

The lamps were switched on and the solutions irradiated for 18 to 24 hours for the ultraviolet (UV) lamps or 72 to 90 hours for the visible (VIS) light lamps. At the end of the selected irradiation time, the cuvettes were removed from the photolysis chamber and mixed by inversion 2-times. The caps were then removed and 40 microliter aliquots taken and diluted with 1 mL of an aqueous solution containing ˜0.9 mg/mL sodium thiosulfate. The thiosulfate solution converted any I₂, IO₃⁻, and I₃⁻ present in the solution tor such that total iodine species could be determined in one assay.

The diluted solutions were assayed by high pressure liquid chromatography (HPLC) using a 4.6×250 mm, C-18, 5 micron particle size, reversed phase column with detection at 280 nm for the contrast agent and 230 nm for the thiosulfate and the iodide ion (r). A mobile phase of 30:70 methanol:aqueous buffer (50 mM KH₂PO₄+7 mL/L 40% tetrabutyl ammonium hydroxide) at 0.75 mL/min was used for elution. Injection volume was 20 microliters. External standards were prepared containing each contrast agent, potassium iodide, and sodium thiosulfate solution and analyzed along with the irradiated samples. The results were reported as weight percentage iodide formed (mg iodide/mg contrast agent times 100).

### Example 3: Determination of UV and Vis Light Exposure in Photolysis Experiments

A 2% solution of quinine hydrochloride dihydrate was prepared in distilled water. The solution was filled into quartz cuvettes, caps were placed on the cuvettes and sealed with Parafilm. One cuvette was wrapped with aluminum foil to serve as a control and one was left unwrapped. The cuvettes were placed under the fluorescent lamps as in Example 2 and exposed to UV light for 39 hours. Over the course of irradiation, the cuvettes were periodically removed from the photolysis chamber and the UV absorbance of the solutions measured at 400 nM using a UV spectrophotometer. The absorbance reading of the control solution was subtracted from the reading of the irradiated solution to give an absorbance due to the light exposure. The control cuvette was re-wrapped with foil and the cuvettes returned to the photolysis chamber. The time during which the cuvettes were not in the light chamber was not included in the measured exposure time. The process was repeated with fresh quinine solution and VIS light exposure for 144 hours.

The absorbance readings at 400 nm due to light exposure were plotted against time of exposure and the resulting correlation used to determine the UV and VIS light exposure. A change in absorbance reading of 0.45 was assumed equivalent to 200 watt hours/square meter for the UV light and a change of 0.51 was assumed equivalent to 1.2 million lux hours for the VIS light. The UV lamps provided 6.62 watts/square meter and the VIS lamps provided 7180 lux.

### Example 4: Effect of Contrast Agent:SCD Mole Ratio on Photostability of Contrast Agents Towards UV Light Irradiation

Aqueous solutions were prepared containing 50 mM contrast agent by diluting commercially available solutions with water. Tromethamine HCl buffer (TRIS), was added to solutions as necessary to increase the TRIS content to that of the commercial product before dilution. Composition of the solutions in the absence of SCD is shown in the following table.

| Contrast | Contrast | | Calcium Disodium | Calcium Chloride | Sodium Chloride |
|---|---|---|---|---|---|
| Agent | Agent (M) | TRIS (mglmL) | Edetate (mglmL) | Dihydrate (mglmL) | (mglmL) |
| Iopamidol | 0.05 | 1.0 | 0.025 | 0 | 0 |
| Iodixanol | 0.05 | 1.2 | 0.0052 | 0.0052 | 0.1320 |
| Ioversol | 0.05 | 3.6 | 0.011 | 0 | 0 |
| Iopromide | 0.05 | 2.42 | 0.0052 | 0 | 0 |
| Ioxaglate | 0.05 | 0 | 0.012 | 0 | 0 |
| Iohexol | 0.05 | 1.21 | 0.00064 | 0 | 0 |

SCD was added to the solutions at various mole ratios and the pH of the solutions adjusted to 7.4 with 0.1N hydrochloric acid or 0.1N sodium hydroxide. The solutions were irradiated with UV light for 18 hours (119 watt hours/m²) then processed, analyzed and reported as in Example 2. The SCDs evaluated were sulfobutyl ether beta-cyclodextrin with an average degree of substitution of 7 (SBE-704CD), and two 2-hydroxypropyl beta-cyclodextrin derivatives having 6.3 (HP-6.3n/CD) and 4.3 (HP-4.3-βCD) average degrees of substitution.

TABLE 1
Iodide degradants formed (wt %) in the absence and presence of varying
amounts and type of SCD at pH 7.4. The percent change in iodide
degradants formed as compared to control is also listed in parentheticals.

| | Contrast: SCD Mole Ratio | | | | | | | | |
|---|---|---|---|---|---|---|---|---|---|
| SCD | 1:0 | 1:0.01 | 1:0.02 | 1:0.025 | 1:0.05 | 1:0.1 | 1:0.5 | 1.1 | 1:2 |
| Iopamidol | | | | | | | | | |
| SBE-7-βCD | 0.553 | 0.532 | n/d | n/d | n/d | 0.476 | 0.393 | n/d | 0.342 |
| | (control) | (−3.8%) | | | | (−13.9%) | (−28.9%) | | (−38.2%) |
| HP-6.3-βCD | 0.553 | 0.547 | n/d | n/d | n/d | n/d | n/d | n/d | 0.394 |
| | (control) | (1.1%) | | | | | | | (−28.7%) |
| HP-4.3-βCD | 0.553 | | n/d | n/d | n/d | n/d | n/d | n/d | 0.439 |
| | (control) | | | | | | | | (−20.6%) |
| Iodixanol | | | | | | | | | |
| SBE-7-βCD | 0.269 | 0.254 | 0.231 | n/d | n/d | n/d | n/d | 0.193 | 0.180 |
| | (control) | (−5.6%) | (−14.15) | | | | | (−28.3%) | (−33.1%) |
| HP-6.3-βCD | 0.269 | n/d | n/d | n/d | n/d | n/d | n/d | n/d | |
| | (control) | | | | | | | | |
| Ioversol | | | | | | | | | |
| SBE-7-βCD | 1.20 | 0.0995 | 0.859 | n/d | n/d | n/d | n/d | 0.498 | 0.387 |
| | (control) | (−17.1%) | (−28.4%) | | | | | (−58.5%) | (−67.8%) |
| HP-6.3-βCD | 1.20 | n/d | n/d | n/d | n/d | n/d | n/d | n/d | 0.783 |
| | (control) | | | | | | | | (−34.8%) |
| Iopromide | | | | | | | | | |
| SBE-7-βCD | 0.672 | 0.483 | 0.407 | n/d | n/d | n/d | n/d | 0.311 | 0.272 |
| | (control) | (−28.1%) | (−28.4%) | | | | | (−53.9%) | (−59.5%) |
| HP-6.3-βCD | 0.672 | n/d | n/d | n/d | n/d | n/d | n/d | n/d | 0.424 |
| | (control) | | | | | | | | (−36.9%) |
| Ioxaglate | | | | | | | | | |
| SBE-7-βCD | 0.282 | 0.242 | 0.228 | n/d | n/d | n/d | n/d | 0.190 | 0.171 |
| | (control) | (−14.2%) | (−19.1%) | | | | | (−32.6%) | (−39.4%) |
| HP-6.3-βCD | 0.282 | n/d | n/d | n/d | n/d | n/d | n/d | n/d | 0.213 |
| | (control) | | | | | | | | (−24.5%) |
| Iohexol | | | | | | | | | |
| SBE-7-βCD | 0.773 | 670 | n/d | n/d | 0.655 | 0.585 | 0.460 | n/d | 0.343 |
| | (control) | (−13.3%) | | | (−15.3%) | (−24.3%) | (−40.5%) | | (−55.6%) |
| HP-6.3-βCD | 0.773 | n/d | n/d | 0.670 | 0.650 | n/d | n/d | n/d | n/d |
| | (control) | | | (−13.3%) | (−15.9%) | | | | |
| HP-4.3-βCD | 0.773 | n/d | n/d | 0.689 | 0.659 | n/d | n/d | n/d | n/d |
| | (control) | | | (−10.9%) | (−14.7%) | | | | |

n/d = not determined

The SCDs stabilized the contrast agents against photolysis by UV irradiation at all mole ratios tested.

### Example 5: Effect of pH and SCD on the Stability of Contrast Agents Following UV Light Irradiation

Aqueous solutions were prepared containing 50 mM contrast agent by diluting commercially available solutions with distilled water. Tromethamine HCl buffer (TRIS), was added to some solutions to increase the buffer content. Composition of the solutions in the absence of SCD is shown in the following table.

| Contrast | Contrast | TRIS | Calcium Disodium | Calcium Chloride | Sodium Chloride |
|---|---|---|---|---|---|
| Agent | Agent (M) | (mglmL) | Edetate (mglmL) | Dihydrate (mglmL) | (mglmL) |
| Iopamidol | 0.05 | 2.42 | 0.025 | 0 | 0 |
| Iodixanol | 0.05 | 1.2 | 0.0052 | 0.0052 | 0.1320 |
| Ioversol | 0.05 | 3.6 | 0.011 | 0 | 0 |
| Iopromide | 0.05 | 2.42 | 0.0052 | 0 | 0 |
| Ioxaglate | 0.05 | 0 | 0.012 | 0 | 0 |
| Iohexol | 0.05 | 2.42 | 0.00064 | 0 | 0 |

The solutions were divided and SBE-7-CD added to one aliquot at a contrast agent:SCD mole ratio of 1:1. The pH of the solutions was adjusted to 5, 6, 7, 7.4 or 8 with 0.1N hydrochloric acid or sodium hydroxide. The solutions were irradiated with UV light for 18 hours (119 watt hours/m2) then processed, analyzed and reported as in Example 2. The results are presented in the table below.

TABLE 2
Iodide degradants formed (wt %) in the absence and presence of SCD at
various starting pH values. The percent change in iodide degradants
formed in the presence of the SCD is also listed.

| Contrast | Agent SCD | pH | | | | |
|---|---|---|---|---|---|---|
| Agent | mole ratio | 5 | 6 | 7 | 7.4 | 8 |
| Iopamidol | 1:0 | 0.366 | 0.373 | 0.626 | 0.670 | 0.829 |
| | 1:1 | 0.265 | 0.282 | 0.373 | 0.434 | 0.761 |
| | % Change | −27.6% | −24.4% | −40.4% | −35.1% | −8.2% |
| Iodixanol | 1:0 | 0.154 | n/d | n/d | 0.269 | 0.342 |
| | 1:1 | 0.139 | n/d | n/d | 0.193 | 0.313 |
| | % Change | −9.7% | n/d | n/d | −28.3% | −8.5% |
| Ioversol | 1:0 | 0.408 | n/d | n/d | 1.20 | 1.46 |
| | 1:1 | 0.230 | n/d | n/d | 0.498 | 0.774 |
| | % Change | −43.6% | n/d | n/d | −58.5% | −47% |
| Iopromide | 1:0 | 0.284 | n/d | n/d | 0.672 | 0.979 |
| | 1:1 | 0.195 | n/d | n/d | 0.311 | 0.470 |
| | % Change | −31.3% | n/d | n/d | −53.9% | −52% |
| Ioxaglate | 1:0 | 0.216 | n/d | n/d | 0.282 | 0.341 |
| | 1:1 | 0.165 | n/d | n/d | 0.190 | 0.272 |
| | % Change | −23.6% | n/d | n/d | −32.6% | −20.2% |
| Iohexol | 1:0 | 0.533 | 0.583 | 0.886 | 1.40 | 2.44 |
| | 1:1 | 0.285 | 0.320 | 0.391 | 0.502 | 1.29 |
| | % Change | −46.5% | −45.1% | −55.9% | −64.1% | −47.1% |

The presence of the SCD stabilized the contrast agents against photolysis at all pH values tested. As shown in Table 2, all compositions containing SCD produced at least 8% less and up to 64% less iodide degradants after exposure to UV light as compared to those composition which contained no SCD.

### Example 6: Effect of Tris Buffer Content and SCD on the Stability of Iopamidol Following UV Light Irradiation

Solutions were prepared containing 100 mM iopamidol, 0.05 mg/mL calcium disodium edetate, varying amounts of tromethamine buffer, and 0 or 5 mM SBE-7-βCD (iopamidol:SCD mole ratio of 1:0.05) in distilled water. The pH of the solutions was adjusted to 7.4 with 0.1N hydrochloric acid. The solutions were irradiated with UV light for 18 hours (119 watt hours/m² then processed, analyzed and reported as in Example 2. Results of the study are presented in the table below.

TABLE 3
Iodide degradants formed (wt %) in the absence
and presence of SCD and various amounts of TRIS buffer.

| | | Iopamidol:SCD | | |
|---|---|---|---|---|
| Starting | TRIS buffer | Mole Ratio | | % Change in iodide |
| pH | content (mM) | 1:01 | 1:0.05 | degradants |
| 7.4 | 5 | 0.875 | 0.746 | −14.7% |
| 7.4 | 7.5 | 0.951 | 0.871 | −8.4% |
| 7.4 | 10 | 1.07 | 0.978 | −8.6% |
| 7.4 | 15 | 1.07 | 1.00 | −6.5% |
| 7.4 | 20 | 1.07 | 1.09 | −3.5% |

The presence of the SCD stabilized the iopamidol solution against photolysis at TRIS buffer concentrations of 5 to 20 mM and an iopamidol:SCD mole ratio of 1:0.05.

### Example 7: Effect of Buffer Type and SCD on the Stability of Iohexol Following UV Light Irradiation

Aqueous solutions were prepared containing 473 mM iohexol and 10 mM tromethamine HCl (TRIS) or sodium phosphate buffer along with the presence or absence of sulfobutyl ether -cyclodextrin (SBE7-β-CD) and disodium calcium edetate. The pH of the solutions was adjusted to 8.0 with 0.1N hydrochloric acid or 0.1N sodium hydroxide. Aliquots of the solutions were irradiated with ultraviolet (UV) light for 24 hours (159 watt hours/m2), then processed, analyzed and reported as in Example 2, with the exception that the iohexol peak was measured at 300 nm for both samples and standards. Results of the study are presented in the table below along with the solution compositions.

TABLE 4
Iodide degradants formed (wt %) in the absence and presence of
SCD and various amounts of TRIS and sodium phosphate buffers.

| Iohexol:SBE7-β- | | Sodium | Calcium Disodium | I⁻ formed % | Change in iodide |
|---|---|---|---|---|---|
| CD Mole Ratio | TRIS (mM) | Phosphate (mM) | Edetate (mglmL) | (wt %) | degradants |
| 1:0 | 10 | — | — | 0.196 | — |
| 1:0.05 | 10 | — | — | 0.128 | −34.7% |
| 1:0.1 | 10 | — | — | 0.120 | −38.8% |
| 1:0 | 10 | — | 0.1 | 0.219 | — |
| 1:0.05 | 10 | — | 0.1 | 0.171 | −21.9% |
| 1:0 | — | 10 | — | 0.539 | — |
| 1:0.05 | — | 10 | — | 0.504 | −6.5% |
| 1:0.1 | — | 10 | — | 0.469 | −13% |
| 1:0 | — | 10 | 0.1 | 0.520 | — |
| 1:0.05 | — | 10 | 0.1 | 0.477 | −8.3% |

The SCD stabilized iohexol against photolysis by UV light in the presence of either tromethamine or sodium phosphate buffer at pH 8. The SCD also stabilized formulations containing the metal chelator disodium calcium edetate.

### Example 8: Effect of CD Ring Size and Substituents on the Stability of Iohexol Following UV and Vis Light Irradiation

Aqueous solutions were prepared containing 473 mM iohexol (180 mg 1/mL), 10 mM tromethamine HCl buffer (TRIS), with and without various SCDs. The pH of the solutions was adjusted to 7.4 with 0.1N hydrochloric acid. Aliquots of the solutions were irradiated with ultraviolet (UV) light for 18 hours (119 watt hours/m²) and other aliquots irradiated with visible (VIS) light for 90 hours (0.65 million lux hours). The solutions were then processed, analyzed and reported as in Example 2, with the exception that the iohexol peak was measured at 300 nm for both samples and standards. Results of the study are presented in the table below along with the SCD used. The SCDs included derivatives prepared from alpha, beta, and gamma CD to evaluate the effect of CD cavity size. Various substituents, including one derivative containing more than one type of substituent, and degrees of substitution (DS) were also evaluated.

TABLE 5

| | | | Average | Iohexol:SCD | I- formed (wt %) | | % Change in |
|---|---|---|---|---|---|---|---|
| SCD | CD Ring | Substituent | DS | Mole Ratio | UV | VIS | iodide degradants |
| None (Control) | — | — | — | — | 0.107 | 0.0617 | — |
| Crsymeb | Beta | Methyl | 4 | 1:0.05 | 0.0969 | 0.0601 | −9.4% (UV) |
| | | | | | | | −2.6%% (Vis) |
| | | | 4 | 1:0.05 | 0.0936 | 0.0577 | −12.5% (UV) |
| | | | | | | | −6.5% (Vis) |
| SBEγCD | gamma | Sulfobutyl | 2.0 | 1:0.05 | 0.101 | 0.0611 | −5.6% (UV) |
| | | | | | | | −.1% (Vis) |
| SBEαCD | alpha | Sulfobutyl | 3.9 | 1:0.05 | 0.0753 | 0.0545 | −5.6% (UV) |
| | | | | | | | −7% (Vis) |
| SPEβCD | beta | Sulfopropyl | 4.0 | 1:0.05 | 0.867 | 0.0574 | −19% (UV) |
| | | | | | | | −7% (Vis) |
| SBE-EE-βCD | Beta | Sulfobutyl | 3.5 | 1:0.05 | 0.0825 | 0.0584 | −22.9% (UV) |
| | | Ethyl | 3.5 | | | | −5.3% (Vis) |
| SBE4.6γCD Control | — | — | — | — | 0.141 | 0.0575 | — |
| SBEγCD | gamma | Sulfobutyl | 4.6 | 1:0.05 | 0.115 | 0.0469 | −18.4% (UV) |
| | | | | | | | −18.4% (vis) |

Each of the SCDs stabilized iohexol against photolysis by both UV and visible light.

### Example 9: Effect of SCDS on the Stability of Contrast Agents Subjected to Thermal and/or Photolytic Stress

Aqueous solutions were prepared containing 50 mM iodinated contrast agent, 10 mM tromethamine HCl buffer (TRIS), with and without sulfobutylether beta-cyclodextrin with an average degree of substitution-7 (SBE7-β-CD), or 2-hydroxypropyl beta-cyclodextrin with an average degree of substitution of ˜6.3 (HP6.3-β-CD) or ˜4.3 (HP4.3-β-CD). The iohexol solution was prepared by dissolving solid iohexol powder while the other solutions were prepared by diluting commercial formulated products and adding sufficient TRIS buffer to reach 10 mM. The pH of the solutions was adjusted to 7.4 with 0.1N hydrochloric acid.

Aliquots of the solutions were irradiated with visible light for 72 hours (0.52 million lux hours). Other aliquots were autoclaved for 20 minutes at 121° C., cooled to room temperature and then irradiated with ultraviolet light for 18 hours (119 watt hours/m2). The solutions were then processed, analyzed and reported as in Example 2, except that the chromatographic peaks of the contrast agents were evaluated at 300 nm for both samples and standards. Results of the study are presented in the tables below.

TABLE 6
Iodide formed (wt %) after irradiation with visible light. The percent
change in iodide degradants formed in the presence of the SCD is also
listed.

| Contrast | | | Agent:SCD Mole Ratio | | | | | | |
|---|---|---|---|---|---|---|---|---|---|
| Agent | Prepared from | SCD | 1:0 | 1:0.01 | 1:0.025 | 1:0.05 | 1:0.1 | 1:0.5 | 1:2 |
| Iohexol | powder | SBE7-β-CD | 0.355 | 0.337 | n/d | 0.316 | 0.313 | 0.274 | 0.183 |
| | | | | (−5.1%) | | (−11%) | (−11.8%) | (−22.8%) | (−48.5%) |
| | | HP6.3- -CD | 0.355 | n/d | 0.346 | 0.341 | n/d | n/d | n/d |
| | | | | | (−2.5%) | (−3.9%) | | | |
| | | HP4.3-β-CD | 0.355 | n/d | 0.331 | 0.324 | n/d | n/d | n/d |
| | | | | | (−6.8%) | (−8.7%) | | | |
| Iopamidol | Isovue ® -M200 | SBE7-β-CD | 0.356 | n/d | n/d | n/d | 0.355 | n/d | n/d |
| | | | | | | | (−0.3%) | | |
| Iodixanol | Visipaque ® 320 | SBE7-β-CD | 0.153 | n/d | n/d | n/d | 0.142 | n/d | n/d |
| | | | | | | | (−7.2%) | | |
| Ioversol | Optiray ™ 350 | SBE7-β-CD | 0.304 | n/d | n/d | n/d | 0.301 | n/d | n/d |
| | | | | | | | (−1%) | | |
| Iopromide | Ultravist ® 370 | SBE7-β-CD | 0.311 | n/d | n/d | n/d | 0.269 | n/d | n/d |
| | | | | | | | (−13.8%) | | |
| Ioxaglate | Hexabrix ® | SBE7-β-CD | 0.231 | n/d | n/d | n/d | 0.211 | n/d | n/d |
| | | | | | | | (−8.6%) | | |

TABLE 7
Iodide formed (wt % × 10³) after autoclaving. The percent change in
iodide degradants formed in the presence of the SCD is also listed.

| Contrast | | | Agent:SCD Mole Ratio | | | | | | |
|---|---|---|---|---|---|---|---|---|---|
| Agent | Prepared from | SCD | 1:0 | 1:0.01 | 1:0.025 | 1:0.05 | 1:0.1 | 1:0.5 | 1:2 |
| Iohexol | powder | SBE7-β-CD | 3.35 | 3.24 | n/d | 2.77 | 4.98 | 4.07 | 2.48 |
| | | | | (−3.3%) | | (−17.3%) | | | (−26%) |
| | | HP6.3-β-CD | 3.35 | n/d | 1.96 | 3.46 | n/d | n/d | n/d |
| | | | | | (−41.5%) | | | | |
| | | HP4.3- β-CD | 3.35 | n/d | 3.00 | 1.43 | n/d | n/d | n/d |
| | | | | | (−10.4%) | (−57.3%) | | | |
| Iopamidol | Isovue ® -M200 | SBE7-β-CD | 3.55 | n/d | n/d | n/d | 1.99 | n/d | n/d |
| | | | | | | | (−43.9%) | | |
| Iodixanol | Visipaque ® 320 | SBE7-β-CD | 5.49 | n/d | n/d | n/d | 3.26 | n/d | n/d |
| | | | | | | | (−40.6%) | | |
| Ioversol | Optiray ™ 350 | SBE7-β-CD | 4.52 | n/d | n/d | n/d | 3.80 | n/d | n/d |
| | | | | | | | (−30.8%) | | |
| Iopromide | Ultravist ® 370 | SBE7-β-CD | 2.96 | n/d | n/d | n/d | 2.48 | n/d | n/d |
| | | | | | | | (−16.2%) | | |
| Ioxaglate | Hexabrix ® | SBE7-β-CD | 1.39 | n/d | n/d | n/d | 1.43 | n/d | n/d |
| | | | | | | | (+2.8%) | | |

TABLE 8
Iodide formed (wt %) after autoclaving then irradiation with ultraviolet
light. The percent change in iodide degradants formed in the presence of
the SCD is also listed.

| Contrast | | | Agent:SCD Mole Ratio | | | | | | |
|---|---|---|---|---|---|---|---|---|---|
| Agent | Prepared from | SCD | 1:0 | 1:0.01 | 1:0.025 | 1:0.05 | 1:0.1 | 1:0.5 | 1:2 |
| lohexol | powder | SBE7-β-CD | 0.730 | 0.693 | n/d | 0.574 | 0.526 | 0.384 | 0.282 |
| | | | | (−5.1%) | | (−21.4%) | (−27.9%) | (−47.4%) | (−61.4%) |
| | | HP6.3-β-CD | 0.730 | n/d | 0.656 | 0.603 | n/d | n/d | n/d |
| | | | | | (−10.1%) | (−17.4%) | | | |
| | | HP4.3-β-CD | 0.730 | n/d | 0.637 | 0.616 | n/d | n/d | n/d |
| | | | | | (−12.7%) | (−15.6%) | | | |
| Iopamidol | Isovue ® -M200 | SBE7-β-CD | 0.540 | n/d | n/d | n/d | 0.470 | n/d | n/d |
| | | | | | | | (−13%) | | |
| Iodixanol | Visipaque ® 320 | SBE7-β-CD | 0.292 | n/d | n/d | n/d | 0.220 | n/d | n/d |
| | | | | | | | (−24.7%) | | |
| Ioversol | Optiray ™ 350 | SBE7-β-CD | 0.665 | n/d | n/d | n/d | 0.545 | n/d | n/d |
| | | | | | | | (−18%) | | |
| Iopromide | Ultravist ® 370 | SBE7-β-CD | 0.487 | n/d | n/d | n/d | 0.330 | n/d | n/d |
| | | | | | | | (−32.3%) | | |
| Ioxaglate | Hexabrix ® | SBE7-β-CD | 0.392 | n/d | n/d | n/d | 0.332 | n/d | n/d |
| | | | | | | | (−15.3%) | | |

TABLE 9
Iodide formed (wt %) after autoclaving then irradiation with visible light.
The percent change in iodide degradants formed in the presence
of the SCD is also listed.

| Contrast | | | Agent:SCD Mole Ratio | | | | | | |
|---|---|---|---|---|---|---|---|---|---|
| Agent | Prepared from | SCD | 1:0 | 1:0.01 | 1:0.025 | 1:0.05 | 1:0.1 | 1:0.5 | 1:2 |
| Iohexol | powder | SBE7-β-CD | 0.392 | 0.392 | n/d | 0.334 | 0.327 | 0.262 | 0.163 |
| | | | | (−0%) | | (−14.8%) | (−16.6%) | (−33.4%) | (-58.4%) |
| | | HP6.3-β-CD | 0.392 | n/d | 0.320 | 0.313 | n/d | n/d | n/d |
| | | | | | (−18.4%) | (−20.2%) | | | |
| | | HP4.3-β-CD | 0.392 | n/d | 0.317 | 0.305 | n/d | n/d | n/d |
| | | | | | (−19.1%) | (−22.2%) | | | |
| Iopamidol | Isovue ® -M200 | SBE7-β-CD | 0.348 | n/d | n/d | n/d | 0.344 | n/d | n/d |
| | | | | | | | (−1.1%) | | |

The SCDs provided stabilization towards degradation by visible light at all mole ratios evaluated. Samples that were autoclaved and then irradiated by ultraviolet light irradiation were also stabilized by SCDs at all mole ratios. The contrast agents that were autoclaved and then irradiated with visible light were stabilized by the SCDs only at agent:cyclodextrin mole ratios greater than 1:0.01.

### Example 10: Effect of Buffer Content and SCD on pH and Stability after Autoclaving and Irradiation with UV or Visible Light

Aqueous solutions were prepared containing 50 mM iohexol, 0, 2.5, 5, 7.5, or 10 mM tromethamine HCl (TRIS) buffer, with and without 2.5 mM sulfobutylether beta-cyclodextrin with an average degree of substitution-7 (SBE7-β-CD). The pH of the solutions was adjusted to 7.4 with 0.1N hydrochloric acid and the solutions were transferred to glass vials. The vials were stoppered, crimp-capped and autoclaved for 20 minutes at 121° C. The pH of the autoclaved solutions was measured after the solutions were at room temperature.

Aliquots of the autoclaved solutions were irradiated with visible light for 72 hours (0.52 million lux hours) and other aliquots irradiated with ultraviolet light for 18 hours (119 watt hours/m2). The solutions were then processed, analyzed and reported as in Example 2, except that the chromatographic peak of the iohexol was evaluated at 300 nm for both samples and standards. Results of the study are presented in the table below.

TABLE 10
Effect of buffer content on the pH and amount of iodide formed (wt %)
after autoclaving or autoclaving plus irradiation with UV or visible
light. The percent change in iodide degradants formed in
the presence of the SCD is also listed.

| | | | pH post | Iodide Formed (wt %) | | |
|---|---|---|---|---|---|---|
| TRIS mM | SBE7-β-CD mM | pH initial | autoclave | Post Autoclave | VIS Irradiation | UV Irradiation |
| 0 | 0 | 7.4 | 6.4 | 0.0151 | 0.465 | 0.444 |
| 2.5 | 0 | 7.4 | 7.3 | 0.00296 | 0.606 | 0.470 |
| 5 | 0 | 7.4 | 7.2 | 0.00283 | 0.672 | 0.510 |
| 7.5 | 0 | 7.4 | 7.2 | 0.00181 | 0.656 | 0.571 |
| 10 | 0 | 7.4 | 7.3 | 0.00086 | 0.662 | 0.646 |
| 0 | 2.5 | 7.4 | 6.6 | 0.00933 | 0.472 | 0.373 (−16.0%) |
| 2.5 | 2.5 | 7.4 | 7.5 | 0.00183 | 0.464 (−23.4%) | 0.368 (−21.7%) |
| 5 | 2.5 | 7.4 | 7.5 | 0.00174 | 0.640 (−4.8%) | 0.421 (−17.4%) |
| 7.5 | 2.5 | 7.4 | 7.4 | 0.00170 | 0.613 (−6.6%) | 0.465 (−18.6%) |
| 10 | 2.5 | 7.4 | 7.4 | 0.00214 | 0.612 (−7.6%) | 0.514 (−20.4%) |

In the absence of a buffer agent, the pH of the iohexol solution dropped after autoclaving by a full pH unit in the absence of the SCD and by 0.8 units in its presence. Less of the iodide degradant was observed in the presence of the SCD after UV irradiation regardless of the amount of buffer present. When a buffer was present, the SCD also stabilized the solutions against photolysis by visible light irradiation.

### Example 11: Effect of SCD on Stability of Concentrated Contrast Agent Solutions after Autoclaving and Irradiation with UV or Visible Light

Aqueous formulations were prepared containing iodinated contrast agents at concentrations used commercially in medical procedures. Sulfobutylether-cyclodextrin, sodium salt, average degree of substitution ˜7 (SBE7CD) was added at varying agent:CD mole ratios. The formulations were placed in glass vials, sealed with rubber stoppers and aluminum crimps, and autoclaved at 121° C. for 20 minutes. Aliquots of the autoclaved solutions were exposed to UV light for 24 hours (159 watt hours/m² or VIS light for 72 hours (0.52 million lux hours) as in Example 2. The solutions were then processed, analyzed and reported as in Example 2, with the following two exceptions; 1) the chromatographic peaks of the contrast agents were evaluated at 300 nm for both samples and standards, and 2) only 20 microliter sample aliquots were taken and diluted for HPLC analysis due to the higher concentrations used in this study. Results of the study are presented in the table below along with the solution compositions.

TABLE 11
Iodide formed (wt %) after autoclaving or autoclaving plus irradiation
with UV or visible light. The percent change in iodide degradants formed
in the presence of the SCD is also listed.

| Contrast | | | CaNa₂ | | | | | |
|---|---|---|---|---|---|---|---|---|
| Agent | Agent:SBE7CD | TRIS | Edetate | CaCl₂•2H₂O | NaCl | Iodide Formed (wt %) | | |
| (mg I/mL) | Mole ratio | (mg/mL) | (mg/mL) | (mg/mL) | (mg/mL) | Autoclaved | UV | VIS |
| Iohexol, pH 7.4 | | | | | | | | |
| 400 | 1:0 | 1.21 | 0.1 | 0 | 0 | 0.00069 | 0.142 | 0.0300 |
| 400 | 1:0.02 | 1.21 | 0.1 | 0 | 0 | 0.00075 | 0.106 | 0.0215 |
| | | | | | | | (−25.4%) | (−28.3%) |
| 400 | 1:0.05 | 1.21 | 0.1 | 0 | 0 | 0.00109 | 0.0958 | 0.0164 |
| | | | | | | | (−32.5%) | (−45.3%) |
| 400 | 1:0.10 | 1.21 | 0.1 | 0 | 0 | 0.00026 | 0.0542 | 0.0065 |
| | | | | | | | (−61.8%) | (−78.3%) |
| 350 | 1:0 | 1.21 | 0.1 | 0 | 0 | 0.00244 | 0.0965 | 0.0391 |
| 350 | 1:0.05 | 1.21 | 0.1 | 0 | 0 | 0.00024 | 0.0564 | 0.0265 |
| | | | | | | | (−41.6%) | (−32.2%) |
| 300 | 1:0 | 1.21 | 0.1 | 0 | 0 | 0.00293 | 0.100 | 0.0417 |
| 300 | 1:0.05 | 1.21 | 0.1 | 0 | 0 | 0.00040 | 0.0717 | 0.0385 |
| | | | | | | | (−28.3%) | (−76.7%) |
| 240 | 1:0 | 1.21 | 0.1 | 0 | 0 | 0.00391 | 0.118 | 0.0536 |
| 240 | 1:0.05 | 1.21 | 0.1 | 0 | 0 | 0.00117 | 0.0928 | 0.0491 |
| | | | | | | | (−21.3%) | (−8.4%) |
| 180 | 1:0 | 1.21 | 0.1 | 0 | 0 | 0.0097 | 0.156 | 0.0503 |
| 180 | 1:0.05 | 1.21 | 0.1 | 0 | 0 | 0.0105 | 0.127 | 0.0476 |
| | | | | | | | (−18.6%) | (−5.4%) |
| 155 | 1:0 | 1.21 | 0.1 | 0 | 0 | 0.00021 | 0.153 | 0.0488 |
| 155 | 1:0.02 | 1.21 | 0.1 | 0 | 0 | 0.00019 | 0.121 | 0.0432 |
| | | | | | | | (−20.9%) | (−11.5%) |
| 155 | 1:0.05 | 1.21 | 0.1 | 0 | 0 | 0.00016 | 0.111 | 0.0417 |
| | | | | | | | (−27.4%) | (−14.5%) |
| 155 | 1:0.10 | 1.21 | 0.1 | 0 | 0 | 0.00016 | 0.0979 | 0.0362 |
| | | | | | | | (−36.0%) | (−25.8%) |
| 140 | 1:0 | 1.21 | 0.1 | 0 | 0 | 0.00474 | 0.191 | 0.0967 |
| 140 | 1:0.05 | 1.21 | 0.1 | 0 | 0 | 0.00287 | 0.151 | 0.0893 |
| | | | | | | | (−20.9%) | (−7.6%) |
| Iopamidol, pH 7 | | | | | | | | |
| 200 | 1:0 | 1.0 | 0.26 | 0 | 0 | 0.00347 | 0.150 | 0.0570 |
| 200 | 1:0.05 | 1.0 | 0.26 | 0 | 0 | 0.00338 | 0.145 | 0.0574 |
| | | | | | | | (−3.3%) | |
| Iopromide, pH 7.4 | | | | | | | | |
| 370 | 1:0 | 2.42 | 0.1 | 0 | 0 | 0.00111 | 0.0989 | 0.0392 |
| 370 | 1:0.05 | 2.42 | 0.1 | 0 | 0 | 0.000720 | 0.0780 | 0.0351 |
| | | | | | | | (−21.1%) | (−10.4%) |
| 300 | 1:0 | 2.42 | 0.1 | 0 | 0 | 0.00145 | 0.110 | 0.0493 |
| 300 | 1:0.05 | 2.42 | 0.1 | 0 | 0 | 0.00118 | 0.102 | 0.0426 |
| | | | | | | | (−7.3%) | (−13.6%) |
| 240 | 1:0 | 2.42 | 0.1 | 0 | 0 | 0.00204 | 0.129 | 0.0504 |
| 240 | 1:0.05 | 2.42 | 0.1 | 0 | 0 | 0.00217 | 0.113 | 0.0413 |
| | | | | | | | (−12.4%) | (−18.0%) |
| 150 | 1:0 | 2.42 | 0.1 | 0 | 0 | 0.00383 | 0.148 | 0.0735 |
| 150 | 1:0.05 | 2.42 | 0.1 | 0 | 0 | 0.00289 | 0.110 | 0.0671 |
| | | | | | | | (−25.7%) | (−8.7%) |
| Ioversol, pH 7.1 | | | | | | | | |
| 350 | 1:0 | 3.6 | 0.2 | 0 | 0 | 0.0124 | 0.118 | 0.0283 |
| 350 | 1:0.05 | 3.6 | 0.2 | 0 | 0 | 0.0120 | 0.0864 | 0.0250 |
| | | | | | | | (−26.8%) | (−11.7%) |
| 320 | 1:0 | 3.6 | 0.2 | 0 | 0 | 0.0130 | 0.123 | 0.0323 |
| 320 | 1:0.05 | 3.6 | 0.2 | 0 | 0 | 0.0127 | 0.0778 | 0.0277 |
| | | | | | | | (−36.7%) | (−14.2%) |
| 300 | 1:0 | 3.6 | 0.2 | 0 | 0 | 0.0135 | 0.134 | 0.0361 |
| 300 | 1:0.05 | 3.6 | 0.2 | 0 | 0 | 0.0129 | 0.111 | 0.0298 |
| | | | | | | | (−17.2%) | (−17.4%) |
| 240 | 1:0 | 3.6 | 0.2 | 0 | 0 | 0.0160 | 0.156 | 0.0454 |
| 240 | 1:0.05 | 3.6 | 0.2 | 0 | 0 | 0.0162 | 0.126 | 0.0400 |
| | | | | | | | (−19.2%) | (−11.9%) |
| 160 | 1:0 | 3.6 | 0.2 | 0 | 0 | 0.0244 | 0.225 | 0.0717 |
| 160 | 1:0.05 | 3.6 | 0.2 | 0 | 0 | 0.0241 | 0.171 | 0.0646 |
| | | | | | | | (−24.0%) | (−9.9%) |
| Ioxaglate, pH 7.0 | | | | | | | | |
| **320 | 1:0 | 0 | 0 | 0 | 0 | 0.00457 | 0.114 | 0.0197 |
| **320 | 1:0.05 | 0 | 0 | 0 | 0 | 0.00329 | 0.0845 | 0.0175 |
| | | | | | | | (−25.9%) | (−11.2%) |
| Iodixanol, pH 7.4 | | | | | | | | |
| 320 | 1:0 | 1.2 | 0.1 | 0.044 | 1.11 | 0.0132 | 0.0751 | 0.0265 |
| 320 | 1:0.05 | 1.2 | 0.1 | 0.044 | 1.11 | 0.0135 | 0.0743 | 0.0251 |
| | | | | | | | (−1.1%) | (−5.3%) |
| 270 | 1:0 | 1.2 | 0.1 | 0.074 | 1.87 | 0.0167 | 0.0845 | 0.0325 |
| 270 | 1:0.05 | 1.2 | 0.1 | 0.074 | 1.87 | 0.0161 | 0.0791 | 0.0288 |
| | | | | | | | (−6.4%) | (−11.4%) |

**contained 393 mg of ioxaglate meglumine and 196 mg of ioxaglate sodium, together providing 320 mg 1/mL. The meglumine salt serves as a buffering agent.

The presence of the SCD stabilized the solutions against degradation by autoclaving in most, but not all, formulations. The SCD stabilized all formulations against degradation from autoclaving+ultraviolet light irradiation. The SCD stabilized all formulations except iopamidol against degradation from autoclaving+visible light irradiation.

### Example 12: Effect of SCDS on the Cardiovascular Electrophysiology and Hemodynamics of Iohexol Following Intra-Arterial Administration

The ionic content of contrast agent formulations can have injurious effects on the heart when the blood in the heart is displaced briefly by the contrast agent. Baath, et al. (Acta Radiologica 31 (1990) Fasc.1 pp 99-104) demonstrated in isolated perfused rabbit hearts that a small amount of sodium (19-38 mM) added to a contrast formulation was beneficial in minimizing decreases in contractile force while 154 mM sodium caused a large decrease in contractile force. The SAE-CDs, SAE-HAE-CDs, and the SAE-AE-CDs all have ionic substituents requiring counterions. The effects of the sodium salt of an SAE-CD were evaluated in an instrumented dog model.

Aqueous formulations were prepared containing 755 mg/mL iohexol without (formulation 1) or with (formulation 2) sulfobutylether β-cyclodextrin, sodium salt (average degree of substitution-7) at an iohexol:SCD mole ratio of 1:0.025. The addition of the SCD added 154 mM sodium to the solution as its counterion. The formulations also contained 0.1 mg/mL edetate calcium disodium hydrate, and 1.2 mg/mL tromethamine buffer, with the pH of each solution adjusted to 7.4 with 1N HCl. The solutions were sterilized by filtration through a 0.22 micron filter.

Each formulation was rapidly injected into the left main coronary artery of an instrumented anesthetized 6-10 month old Beagle dog as 5 doses of 4 mL each, administered at ˜1 mL/sec with 10 seconds between doses. Thirty minutes after the last dose, the procedure was repeated with the second formulation. The overall process was repeated in two additional animals.

The instrumentation provided measurement of right heart pressure, left ventricular pressure, aortic pressure, cardiac output and electrocardiography (ECG). The specific parameters measured were: systolic artery pressure (SAP), mean aortic pressure (MAP), diastolic aortic pressure (DAP), left ventricular systolic pressure (LVSP), left ventricular end diastolic pressure (LVEDP), left ventricle dP/dtₘₐₓ (an indirect measure of contractile force), left ventricle dP/dtₘᵢₙ, cardiac output (CO), systemic vascular resistance (SVR), pulmonary vascular resistance (PVR), pulmonary artery pressure (PAP), monophasic action potential duration (MAPD_{30%, 50%, 90%}), heart rate (HR), and from the ECG; PR interval, QRS duration, QT/QTc interval, JT/JTc interval (as needed) and arrhythmogenesis.

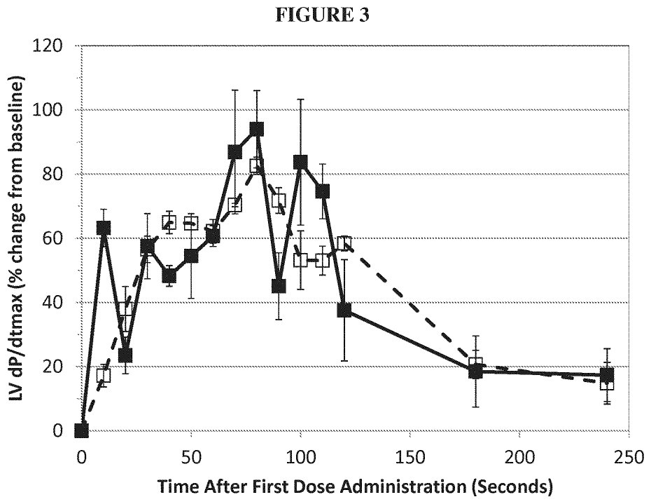

Results: There were no notable effects of intracoronary iohexol administration (formulation 1) on most measured cardiovascular parameters. Variables including LV contractility and QTc interval were notably, yet transiently, altered following the iohexol regimen. Results for QTc interval and contractility are shown in FIGS. 2 and 3 respectively.

In addition to these transient quantitative changes, qualitative alterations in electrocardiographic morphology were observed. These were generally concomitant with physical injection of the iohexol solution into the coronary artery, and likely associated with brief myocardial ischemia from interruption of arterial flow. The changes consisted of QRS complex widening along with ST segment depression. Scattered premature ventricular contractions were also noted. ECG morphology returned to normal within five minutes after the last iohexol injection.

Administration of formulation 2 containing the SCD gave similar quantitative and qualitative changes as formulation 1 demonstrating that the additional sodium content provided by the SCD surprisingly had no detrimental effect on cardiac function.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except by the appended claims. All of the embodiments disclosed and claimed herein can be made and executed without undue experimentation in light of the present disclosure.

1-79. (canceled)

80. An aqueous pharmaceutical composition comprising:
an iodinated contrast agent selected from the group consisting of iopamidol, ioversol, iopromide, and ioxaglate;
a pharmaceutically acceptable buffering agent; and
a substituted cyclodextrin present at a contrast agent:substituted cyclodextrin mole ratio from 1:0.01 to 1:2;
wherein the composition has a pH of 5 to 8; and
wherein the amount of iodine species produced by degradation of the contrast agent is reduced upon exposure of the composition to UV or visible light as compared to a corresponding composition of the contrast agent not having a substituted cyclodextrin.

81. The composition of claim 80, wherein the composition is heat sterilized prior to exposure to the UV or visible light.

82. The composition of claim 81, wherein the heat sterilization comprises steam sterilization.

83. The composition of claim 82, wherein the steam sterilization occurs at 115-116° C. for at least 30 minutes, 121-123° C. for at least 15 minutes, 126-129° C. for at least 10 minutes, or 134-138° C. for at least 3 minutes.

84. The composition of claim 80, wherein the pharmaceutically acceptable buffering agent is selected from the group consisting of tromethamine, phosphate, and meglumine, and their pharmaceutically acceptable salts.

85. The composition of claim 80, wherein the substituted cyclodextrin is selected from the group consisting of sulfoalkylether cyclodextrins, 2-hydroxypropyl cyclodextrins, partially methylated cyclodextrins, and sulfoalkylether alkylether cyclodextrins.

86. The composition of claim 80, wherein the composition exhibits at least a least 5%, 10%, 20%, 30%, 40%, 50%, or 60% reduction in formation of iodine species when exposed to ultraviolet light as compared to the corresponding composition.

87. The composition of claim 80, wherein the composition exhibits at least a 20% reduction in formation of iodine species when exposed to ultraviolet light as compared to the corresponding composition.

88. The composition of claim 80, wherein the composition exhibits at least a 40% reduction in formation of iodine species when exposed to ultraviolet light as compared to the corresponding composition.

89. The composition of claim 80, wherein the light is visible light, and the mole ratio of the contrast agent to the substituted cyclodextrin is about 1:0.1.

90. The composition of claim 80, wherein the composition exhibits at least a least 5%, 10%, 20%, 30%, 40%, 50%, or 60% reduction in formation of iodine species when exposed to visible light as compared to the corresponding composition.

91. A ready to use, sterile, injectable aqueous pharmaceutical composition comprising:
an iodinated contrast agent selected from the group consisting of iopamidol, ioversol, iopromide, and ioxaglate;
1 to 4 mg/mL tromethamine (TRIS) buffer;
0.1 to 0.6 mg/mL disodium calcium edetate; and
a substituted cyclodextrin selected from the group consisting of sulfobutylether beta-cyclodextrin, and 2-hydroxypropyl beta-cyclodextrin, wherein the substituted cyclodextrin is present at a contrast agent:substituted cyclodextrin mole ratio from 1:0.01 to 1:0.1;
wherein the composition has a pH of 5 to 8 and wherein the composition is packaged in a primary container that does not possess enhanced light shielding properties.

92. The composition of claim 91, wherein the composition has been heat sterilized.

93. The composition of claim 91, wherein the iodinated contrast agent is present at a molar concentration greater than 394.1 mM and less than or equal to 1051 mM.

94. The composition of claim 91, wherein the iodinated contrast agent is iodixanol, and the iodixanol is present at a molar concentration greater than 197.1 mM and less than or equal to 525.4 mM.

95. The composition of claim 91, further comprising one or more components selected from the group consisting of pH adjusting agents, antioxidants, chelating agents, and inert gasses.

96. The composition of claim 91, wherein the pH of the composition is 6.5 to 7.7.

97. The composition of claim 91, wherein the sulfobutylether beta-cyclodextrin has an average degree of substitution of 7.

98. The composition of claim 91, comprising 1.0 to 1.21 mg/ml tromethamine and 0.1 to 0.48 mg/ml disodium calcium edetate.
